(19) United States
(12) Patent Application Publication
(10) Pub. No.: US 2026/0216257 A1
(43) Pub. Date: Jul. 30, 2026

(54) TUMOR-TARGETING PHARMACEUTICAL COMBINATIONS

(71) Applicants: YEDA RESEARCH AND DEVELOPMENT CO. LTD., REHOVOT (IL); BACCINE LTD., REHOVOT (IL)

(72) Inventors: Ravid STRAUSSMAN, Mazkeret Batia (IL); Oded SANDLER, Mazkeret Batya (IL); Deborah Gitta ROSENBERG, Petach Tikva (IL)

(21) Appl. No.: 19/444,024

(22) Filed: Jan. 8, 2026

(30) Foreign Application Priority Data
Jan. 8, 2025 (IL) 318256

(51) Int. Cl. A61K 35/74; A61K 35/00; A61K 39/00; A61K 39/02; A61P 37/04

(52) U.S. Cl. CPC A61K 35/74; A61K 39/0011; A61K 39/02; A61P 37/04; A61K 2035/11; A61K 2039/585

(57) ABSTRACT

A pharmaceutical combination and uses thereof, wherein the pharmaceutical combination comprises a tumor-homing bacteria and a host-transient bacterial adjuvant comprising a supplementary dose of an immunostimulant.

This application claims the benefit of Israeli Patent Application No. 318256, filed 8 Jan. 2025, the contents of which are incorporated herein in their entirety.

Provided herein are pharmaceutical combinations comprising an immunostimulant and a tumor-homing bacteria, and their use in a method for the prevention, delay of progression, or treatment of cancer in a subject. Also provided herein are pharmaceutical combinations and methods for use in modulating an immune system response in a host with cancer while providing a tumor-homing bacteria exhibiting human serum resilience. Further provided herein are pharmaceutical combinations comprising a bacterium genetically modified for transient synthesis of an immunostimulant and a tumor-homing bacteria, and their use in a method for the prevention, delay of progression, or treatment of cancer in a subject.

Parenteral bacterial cancer therapies that are designed for tumor colonization need to overcome systemic adverse effects associated with parenteral administration of bacteria. These systemic adverse effects are related to unacceptable toxicity or pyrogenicity in the host due to exposure to bacterial endotoxins which are believed to stimulate both direct and indirect production of various host factors.

Pathogen-associated molecular patterns (PAMPs) are endotoxins or potent immunostimulants, such as lipopolysaccharide (LPS), typically found on the surface of Gram-negative bacteria. Toll-like receptors (TLRs) are pattern recognition receptors that recognize molecular patterns associated with pathogens and play a crucial role in modulating a host immune response. Upon activation by immunostimulants such as LPS, for example, TLRs initiate a signaling cascade which mounts a defense against invading pathogens. The interplay between these elements forms the cytokine cascade, with TNFα identified as a crucial component. Early mediators in this process are thought to initiate the release of subsequent factors.

Many methods for mitigating the systemic toxicity associated with exposure to bacteria have been examined. Disrupting genes involved in immune-stimulating PAMPs and reduced activation of the complement system collectively aim to address systemic toxicity by minimizing the inflammatory response during parenteral administration.

For example, U.S. Pat. No. 6,863,894 discloses a Gram-negative bacterial strain, Salmonella typhimurium VNP20009, which is a tumor-targeted bacteria attenuated for safety by deleting both the msbB and purl genes. Bioengineering LPS by deletion of msbB results in a less toxic form of Lipid A, while deleting the purl gene renders the bacteria auxotrophic for purines, which are present in the interstitial fluid of tumors. The reduced toxicity provided by inducing only 10 to 35 percent of the TNFα induced by a wild-type S. typhimurium enabled dosing in mice of 10⁶ CFU per mouse. Although there was evidence of tumor colonization at the maximum tolerated dose (MTD), it was not possible to test higher doses, which are assumed to be required to affect anti-tumor activity, due to toxicity that correlated with high levels of pro-inflammatory cytokines.

Translating preclinical data into a clinical setting has presented notable challenges, primarily attributed to poor tumor colonization observed in human melanoma tumors within the framework of the MTD of 3×10⁸ CFU/m². Higher doses were not possible due to toxicity correlated with high levels of pro-inflammatory cytokines.

Since then, additional strategies have been developed to minimize systemic toxicity and increase tumor and/or tumor microenvironment colonization.

For example, U.S. Patent Application No. 2022/0047649 discloses killed or non-viable Gram-negative organisms with substantially reduced endotoxin activity due to exposure to antibiotic polymyxin B or glutaraldehyde which inactivates LPS activity.

U.S. Pat. No. 11,779,612 discloses a human serum resilient strain which is less inflammatory in humans compared with VNP20009. It shows that improved tolerability and decreased immunogenicity in strains that have attenuations in one or both of flagellin and PagP, or preferably all three of PagP, flagellin, and ASD, demonstrated favorable tolerability and lower immunogenicity while providing an increase in tumor or tumor microenvironment colonization.

However, existing methods continue to encounter difficulty in providing effective tumor-homing and anti-cancer activity while reducing endotoxicity. For example, it was previously discovered that intravenous administration of tumor-homing bacteria genetically modified for synthesis of a toxicity-reduced immunostimulant (by incorporation of the Lipid A 1-phosphatase (lpxE) gene into the bacteria) provided enhanced safety measures in terms of cytokine production and bacterial clearance from the blood in mouse cancer models; however, it also resulted in occasional mouse death. See, e.g., Kong et al. “Phosphate Groups of Lipid A are Essential for Salmonella enterica Serovar Typhimurium Virulence and Affect Innate and Adaptive Immunity.” Infect Immun 2012. 80 (9): 3215-24. doi: 10.1128/IAI.00123-12; and Kong et al. “Salmonella synthesizing 1-dephosphorylated [corrected] lipopolysaccharide exhibits low endotoxic activity while retaining its immunogenicity.” J Immunol 2011. 187 (1): 412-23. doi: 10.4049/jimmunol.1100339. This was the case even when the tumor-homing bacteria were genetically modified to express a tumor-specific antigen. Further, although there was a high rate of tumor-homing with a low dose of the bacteria, treatment efficacy in terms of tumor reduction was reduced.

Although not wishing to be bound by theory, it is believed that these results underline the need for harnessing the positive effects of a supplementary immunostimulant (e.g., LPS) to enhance the efficacy of tumor-homing bacteria. Reducing toxicity by attenuation is a common approach for administering tumor-homing bacteria which naturally express immunostimulants; however, attenuation of immunostimulants may be associated with insufficient tumor colonization in humans and/or reduced efficacy of treatment due to enhanced vulnerability to the human anti-bacterial immune response.

This application is related to International Patent Application No. PCT/IL2022/050191 filed on Feb. 17, 2022, entitled “Genetically modified bacteria for generating vaccines,” International Patent Application No. PCT/IL2023/050876, filed on Aug. 17, 2023, entitled “Genetically modified bacteria for generating vaccines,” and PCT/IB2024/058038, filed on Aug. 19, 2024, entitled “Genetically modified bacteria for multi-modal secretion.” The bacteria provided in each of these applications may be modified as described in this application, and such bacteria are incorporated by reference herein. The subject matter of each of these applications is incorporated by reference in its entirety.

Thus, an unmet need exists for effective bacterial cancer therapies that provide robust, non-toxic immune activation while enhancing tumor colonization at tolerable doses.

### SUMMARY

In the context of bacterial-based cancer therapy, it has now unexpectedly been found that a pharmaceutical combination comprising a dose of tumor-homing bacteria; and a host-transient bacterial adjuvant comprising a supplementary dose of an immunostimulant, or a derivative or precursor thereof, is useful for enhancing efficacy of tumor-homing bacteria while providing tolerable safety measures in terms of cytokine production and bacterial clearance from the blood in mouse cancer models.

The inventors set out to provide induction of a priming inflammatory or innate immune response by an immunostimulant without affecting the vitality of bacteria in human serum which may be associated with tumor homing and colonization in humans. In some embodiments, the immunostimulant has reduced toxicity. More specifically, the inventors provide a pharmaceutical combination comprising a tumor-homing bacteria exhibiting sufficient survival in a human serum environment, wherein the pharmaceutical combination provides a desirable post-treatment profile including the following features: (1) modulated cytokine production (TNF alpha and IL6) in the blood stream of a host organism (e.g., mouse or human); (2) a notable decrease in bacteremia at a delayed, post-administration time point; (3) negligible mortality rates observed in murine hosts; and (4) tolerable impact on body weight, including both loss and subsequent recovery in murine hosts.

The present inventors have surprisingly discovered a pharmaceutical combination which incorporates the desired and contradictory effects of an immunostimulant. The immunostimulant is associated with raised toxicity level in a mammalian host while also contributing to the ability of a bacteria to survive in a mammalian host, which may be associated with successful tumor homing in humans. The inventors have discovered a combination of components, wherein each component addresses a contradictory but desired effect. One component comprises a host-transient supplementary dose of an immunostimulant. The supplementary dose may be transient in a host by a variety of mechanisms, e.g., by transient toxicity, transient immune activation, or transient resilience in a host. The second component comprises a tumor-homing bacteria exhibiting survival in a mammalian host and consistent synthesis of an immunostimulant. In some embodiments, the immunostimulant exhibits reduced toxicity.

In one aspect, there is provided a method of modulating a host immune system and enhancing tumor homing comprising a pharmaceutical combination described herein. Also provided is a pharmaceutical combination for use in modulation of a host immune system and human serum resilience for tumor-homing. Specifically, there is provided a pharmaceutical combination comprising: a tumor-homing bacteria and an immunostimulant, or a derivative or precursor thereof. In some embodiments, the pharmaceutical combination further comprises one or more physiologically acceptable carriers for parenteral administration. In some embodiments, the derivative of the immunostimulant is a toxicity-reduced derivative.

In various aspects, there is provided herein a pharmaceutical combination comprising a dose of tumor-homing bacteria that is at least 50% less than the tumor-homing bacteria's MTD and a host-transient bacterial adjuvant comprising a supplementary dose of an immunostimulant, or a derivative or precursor thereof. In some embodiments, the pharmaceutical combination further comprises one or more physiologically acceptable carriers for parenteral administration. In some embodiments, the derivative of the immunostimulant is a toxicity-reduced derivative.

In some embodiments, the tumor-homing bacteria exhibits constitutive toxicity in a host. In some embodiments, the tumor-homing bacteria exhibits human serum resilience at 3 hours after parenteral administration of more than 3-fold relative to Salmonella typhimurium VNP20009.

In some embodiments, the dose of tumor-homing bacteria is at least 30% less than a maximum tolerated dose of Salmonella typhimurium VNP20009.

In some embodiments, the host-transient bacterial adjuvant is sufficient to provide an endotoxicity increase of at least 30% relative to the dose of the tumor-homing bacteria. In some embodiments, the host-transient bacterial adjuvant is sufficient to provide an endotoxicity increase of at least 50% relative to the dose of the tumor-homing bacteria. In some embodiments, the host-transient bacterial adjuvant is sufficient to provide an endotoxicity increase of at least 70% relative to the dose of the tumor-homing bacteria. In some embodiments, the host-transient bacterial adjuvant is sufficient to provide an endotoxicity increase of at least 75% relative to the dose of the tumor-homing bacteria. In some embodiments, the host-transient bacterial adjuvant is sufficient to provide an endotoxicity increase of at least 80% relative to the dose of the tumor-homing bacteria. In some embodiments, the host-transient bacterial adjuvant is sufficient to provide an endotoxicity increase of at least 85% relative to the dose of the tumor-homing bacteria. In some embodiments, the host-transient bacterial adjuvant is sufficient to provide an endotoxicity increase of at least 90% relative to the dose of the tumor-homing bacteria. In some embodiments, the host-transient bacterial adjuvant is sufficient to provide an endotoxicity increase of at least 95% relative to the dose of the tumor-homing bacteria.

In some embodiments, the host-transient bacterial adjuvant exhibits a transient toxicity in a host blood stream as measured by a return of a toxicity measure to basal levels in a host at least 6 hours post administration.

In some embodiments, the supplementary dose is at least 15% of the maximum tolerated dose of the immunostimulant, derivative, or precursor thereof.

In some embodiments, the host-transient bacterial adjuvant is a bacteria-derived particle comprising an immunostimulant. In some embodiments, the host-transient bacterial adjuvant is an inactivated bacteria comprising an immunostimulant. In some embodiments, the host-transient bacterial adjuvant is a live bacteria comprising an immunostimulant configured to be transiently present in the blood stream of a host. In some embodiments, the host-transient bacterial adjuvant is an immunoactive-transient-bacteria.

In some embodiments, the derivative of the immunostimulant is a toxicity-reduced derivative of the immunostimulant.

In some embodiments, the pharmaceutical combination further comprises one or more physiologically acceptable carriers.

In various embodiments, the present disclosure provides a pharmaceutical combination comprising one or more physiologically acceptable carriers and two bacterial strains. In some embodiments, the two bacterial strains comprise an immunoactive-transient-bacteria and a tumor-homing bacteria. In some embodiments, the tumor-homing bacteria is an immunoactive-constitutive-bacteria. In some embodiments, the immunoactive-transient-bacteria is genetically modified for transient survival in human serum. In some embodiments, the immunoactive-transient-bacteria is genetically modified for inducible synthesis of an immunostimulant. In some embodiments, the immunoactive-transient-bacteria is genetically modified for transient synthesis of an immunostimulant.

In some embodiments, the pharmaceutical combination comprises one or more physiologically acceptable carriers and two bacterial strains:
(a) an immunoactive-transient-bacteria genetically modified for transient survival in human serum, inducible synthesis of an immunostimulant, or transient synthesis of an immunostimulant; and
(b) a tumor-homing bacteria which is an immunoactive-constitutive-bacteria.

In some embodiments, the immunoactive-transient-bacteria is genetically modified for transient synthesis of an immunostimulant. In some embodiments, the genetic modification comprises (i) a deletion or attenuation of a gene encoding a protein involved in synthesis of the immunostimulant; and (ii) insertion of a gene encoding a protein involved in synthesis of the immunostimulant genetically associated with a mechanism for transient synthesis of the immunostimulant.

In some embodiments, the mechanism for transient synthesis of the immunostimulant is an inducible prokaryotic promoter.

In some embodiments, the mechanism for transient synthesis comprises leaky expression of the immunostimulant when not exposed to an inducer.

In some embodiments, the immunoactive-transient-bacteria is an immunoactive inducible bacterial strain and the pharmaceutical combination does not comprise an inducer or comprises trace amounts of an inducer.

In some embodiments, the immunostimulant is a pathogen-associated molecular pattern (PAMP) associated with bacteria.

In some embodiments, the immunostimulant is a Toll-like receptor 2 (TLR2) agonist, a Toll-like receptor 4 (TLR4) agonist, a Toll-like receptor 5 (TLR5) agonist, a Toll-like receptor 3 (TLR3) agonist, a Toll-like receptor 1 (TLR1) agonist, a Toll-like receptor 7 (TLR7) agonist, a complement activator, or a TNFα and/or IFN-γ secretion inducer.

In some embodiments, the tumor-homing bacteria is oncolytic.

In some embodiments, the tumor-homing bacteria comprises intact flagella.

In some embodiments, the combination does not comprise an inducer.

In some embodiments, the tumor-homing bacteria is genetically modified for reduced toxicity of an immunostimulant.

In some embodiments, the immunoactive-transient-bacteria is genetically modified for reduced toxicity of an immunostimulant.

In some embodiments, the genetic modification for reduced toxicity of the immunostimulant comprises attenuated activity in a host of TLR4, TLR3, TLR1, TLR7, TLR5, or TLR2; and/or attenuated TNFα secretion, IFN-γ secretion, and/or complement activation, compared to activity in the host of a bacteria without the genetic modification. In some embodiments, the genetic modification for reduced toxicity of the immunostimulant comprises attenuated activity in a host of TLR4 and/or TLR5.

In some embodiments, the genetic modification for reduced toxicity of the immunostimulant comprises a modification, deletion, and/or insertion of a gene selected from: pagE, pagP, pagL, lpxM, lpxL, lpxE, lpxR, lpxF, lpxXL, and lpxT. In some embodiments, the genetic modification comprises a modification, deletion, and/or insertion of lpxL. In some embodiments, the genetic modification comprises a modification, deletion, and/or insertion of lpxE. In some embodiments, the genetic modification comprises a modification, deletion, and/or insertion of lpxF.

In some embodiments, the genetic modification for reduced toxicity of the immunostimulant comprises a genetic modification of a gene related to biosynthesis of the immunostimulant.

In some embodiments, the biosynthesis comprises a post-synthesis toxicity reduction by structural modification of the immunostimulant compared with a bacterium without the genetic modification.

In some embodiments, the immunostimulant is a cell surface protein. In some embodiments, the immunostimulant comprises Lipid A, lipopolysaccharide (LPS), flagellin, lipoprotein, fimbriae, peptidoglycan, lipoteichoic acid (LTA), core oligosaccharide, or O-antigen. In some embodiments, the immunostimulant comprises Lipid A. In some embodiments, the immunostimulant comprises LPS.

In some embodiments, the tumor-homing bacteria is a Gram-negative bacterium. In some embodiments, the inactivated bacteria comprising an immunostimulant, the live bacteria comprising an immunostimulant configured to be transiently present in the blood stream of a host, and/or the immunoactive-transient-bacteria is a Gram-negative bacterium; or the bacteria-derived particle comprising an immunostimulant is derived from a Gram-negative bacterium.

In some embodiments, the Gram-negative bacterium is a member of a genus selected from: Salmonella, Escherichia, Helicobacter, Neisseria, Yersinia, and Pseudomonas. In some embodiments, the Gram-negative bacterium is a member of the Salmonella genus and is a Salmonella typhimurium having an attenuated or deleted stm3120 gene.

In some embodiments, the tumor-homing bacteria and/or the bacterial adjuvant is further genetically modified to express one or more tumor antigens and/or one or more immunomodulators. In some embodiments, the one or more tumor antigens comprises a neoantigen.

In some embodiments, the tumor-homing bacteria and/or the immunoactive-transient-bacteria is further genetically modified to express one or more tumor antigens and/or one or more immunomodulators. In some embodiments, the one or more tumor antigens comprises a neoantigen.

In some embodiments, the immunoactive-transient-bacteria is present at a higher dose than the tumor-homing bacteria. In some embodiments, the dose of the immunoactive-transient-bacteria relative to the dose of the tumor-homing bacteria is at least 8:1. In some embodiments, the dose of the immunoactive-transient-bacteria relative to the dose of the tumor-homing bacteria is at least 10:1. In some embodiments, the dose of the immunoactive-transient-bacteria relative to the dose of the tumor-homing bacteria is at least 20:1. In some embodiments, the dose of the immunoactive-transient-bacteria relative to the dose of the tumor-homing bacteria is at least 30:1. In some embodiments, the dose of the immunoactive-transient-bacteria relative to the dose of the tumor-homing bacteria is at least 50:1. In some embodiments, the dose of the immunoactive-transient-bacteria relative to the dose of the tumor-homing bacteria is at least 100:1. In some embodiments, the dose of the immunoactive-transient-bacteria relative to the dose of the tumor-homing bacteria is at least 200:1. In some embodiments, the dose of the immunoactive-transient-bacteria relative to the dose of the tumor-homing bacteria is at least 250:1.

In some embodiments, the dose of the immunoactive-transient-bacteria relative to the dose of the tumor-homing bacteria is from 500:1 to 10:1. In some embodiments, the dose of the immunoactive-transient-bacteria relative to the dose of the tumor-homing bacteria is from 400:1 to 20:1. In some embodiments, the dose of the immunoactive-transient-bacteria relative to the dose of the tumor-homing bacteria is from 300:1 to 30:1.

In some embodiments, the dose of tumor-homing bacteria and the host-transient bacterial adjuvant are in separate compositions. In some embodiments, the tumor-homing bacteria and the immunoactive-transient-bacteria are in separate compositions.

In some embodiments, the pharmaceutical combination is provided in a kit.

In some embodiments, provided herein is a method of modulating an immune system response and enhancing tumor-homing in a subject, comprising parenteral administration of any pharmaceutical combination disclosed herein.

In some embodiments, the tumor-homing bacteria and the immunoactive-transient-bacteria are simultaneously administered.

In some embodiments, the parenteral administration is by an intratumoral route. In some embodiments, the parenteral administration is by an intra-peritoneal route. In some embodiments, the parenteral administration is by an intravenous route.

In some embodiments, both the tumor-homing bacteria and the immunoactive-transient-bacteria are administered by intravenous route. In some embodiments, the tumor-homing bacteria is administered by intratumoral route, and the immunoactive-transient-bacteria is administered by intravenous route.

In some embodiments, the immunoactive-transient-bacteria is an immunoactive inducible strain. In some embodiments, the pharmaceutical combination does not comprise an inducer or comprises trace amounts of an inducer.

In some embodiments, the method comprises prevention, delay of progression, or treatment of a cancer.

In some embodiments, the method further comprises administering a second agent or treatment. In some embodiments, the second agent or treatment is an anti-cancer agent or treatment. In some embodiments, the anti-cancer agent or treatment is an immunotherapy.

In some embodiments, provided herein is a method of modulating an immune system response and enhancing tumor-homing, comprising administering to a host circulatory system a tumor-homing bacteria constitutively expressing an immunostimulant, or a derivative or precursor thereof; and a host-transient bacterial adjuvant comprising a supplementary dose of an immunostimulant, or a derivative or precursor thereof.

In some embodiments, provided herein is a method of preventing, delaying the progression of, or treating a cancer, comprising administering to a host circulatory system a tumor-homing bacteria constitutively expressing an immunostimulant, or a derivative or precursor thereof; and a host-transient bacterial adjuvant comprising a supplementary dose of an immunostimulant, or a derivative or precursor thereof.

### BRIEF DESCRIPTION OF THE DRAWINGS

FIG. 1A shows ex vivo evaluation of bacterial-mediated induction of TNFα and IL6 expression in pg/mL in whole blood from healthy human donors for the indicated bacterial strains, including S. typhimurium VNP20009 and various bacterial strains genetically modified for reduced toxicity of LPS and/or induced synthesis of immunostimulating LPS. FIG. 1B shows serum resilience of the indicated bacterial strains, including S. typhimurium VNP20009 and various bacterial strains genetically modified for reduced toxicity of LPS and/or induced synthesis of immunostimulating LPS as measured in CFU/mL.

FIG. 2A shows tissue presence (in CFU/gram) of the indicated bacterial strains, including S. typhimurium VNP20009 bacteria and various bacteria genetically modified for reduced toxicity of LPS and/or induced synthesis of immunostimulating LPS. FIG. 2B shows % weight loss over time upon administration of various bacteria genetically modified for reduced toxicity of LPS and/or induced synthesis of immunostimulating LPS. FIGS. 2C, 2D, and 2E show % weight loss over time upon administration of various bacteria genetically modified for reduced toxicity of LPS and/or induced synthesis of immunostimulating LPS.

FIG. 3 shows various safety measures and human serum resilience of the indicated bacterial strains, including S. typhimurium VNP20009 and multiple bacterial strains genetically modified for reduced toxicity of LPS and/or induced synthesis of immunostimulating LPS.

FIGS. 4A, 4B, 4C, and 4D illustrate tumor growth in tumor-bearing mice treated with various bacterial strains genetically modified for reduced toxicity of LPS and/or induced synthesis of immunostimulating LPS. “Mix”=a mixture of LPS-attenuated bacteria in induced state rfaD with toxicity reduction provided by lpxE (Pac-1-IrfaD/pxE) and tumor-homing bacteria with toxicity reduction provided by lpxE (Pac-1-lpxE).

FIG. 5A shows the weight loss relative to day 0 (injection day) in mice following the indicated bacterial injection. FIG. 5B shows the weight loss relative to day 0 (injection day) in mice following bacterial injection (3 million CFU of Pac-1-IrfaD/lpxE+0.1 million CFU of of Pac-1-lpxE) in various types of tumor models.

FIG. 6A shows the weight loss relative to day 0 (injection day) in mice following bacterial injection (Pac-1 backbone (no neoantigen, no LPS attenuation)) at various doses. FIG. 6B shows colonization of the indicated sites in mice administered various doses of Pac-1-backbone bacterial injection. T=tumor; Li=liver; Sp=spleen.

FIGS. 7A and 7B show tumor volume (FIG. 7A) and percent survival (FIG. 7B) of MC38 tumor-bearing mice injected with the indicated doses of Pac-1 variant (with neoantigen, 1PL, LPS wt).

FIG. 8 shows efficacy of a combination of low dose (10⁵ CFU) of Pac-1-lpxE tumor-homing bacteria supplemented with 10 μg (protein mass) of outer membrane vesicles (OMVs) generated from Pac-1-lpxE bacteria, serving as host-transient immunostimulant in MC38 tumor bearing mice. The average tumor volume per group of mice is shown as indicated.

FIG. 9 shows efficacy of a combination of low dose (10⁵ CFU) of Pac-1-lpxE tumor-homing bacteria supplemented with 50 μg/kg LPS, serving as host-transient immunostimulant in MC38 tumor bearing mice. The average tumor volume per group of mice is shown as indicated.

### DETAILED DESCRIPTION

### Definitions

For the purposes of interpreting this specification, the following definitions will apply and whenever appropriate, terms used in the singular will also include the plural and vice versa. Such terms are to be given their ordinary meaning in the art unless otherwise indicated. Other specifically defined terms are to be construed in a manner consistent with the definitions provided herein. It is to be understood that the terminology used herein is for the purpose of describing particular embodiments only and is not intended to be limiting.

The features, integers, characteristics, and compounds elucidated in association with a specific aspect, embodiment, or illustration of the present invention are deemed to be applicable across all other aspects, embodiments, or examples detailed within this specification. This inclusivity persists unless such application would result in incompatibility. The entirety of the features outlined in this specification (encompassing any associated claims, abstracts, and drawings), along with each step described therein for executing a method or process, may be amalgamated in various combinations. However, it is to be noted that certain combinations may be excluded if they involve mutually exclusive features or steps. The scope of this invention extends beyond the specifics provided within any particular embodiments disclosed herein.

The features, integers, and characteristics outlined in this document pertain to the specific embodiment or example presented. However, these aspects are also applicable to any other embodiments or examples unless they conflict with each other. The disclosed features within this specification (including associated claims, abstract, and illustrations), as well as all described methods or processes, may be integrated in various combinations. It is important to note that certain combinations might not be feasible if the features or steps are mutually exclusive. This invention extends beyond the specific embodiments illustrated herein.

The singular forms “a,” “an,” and “the” include plural referents unless the content clearly dictates otherwise.

The term “about” in the context of numerical values and ranges refers to values or ranges that approximate or are close to the recited values or ranges such that the embodiment may perform as intended, as is apparent to the skilled person from the teachings contained herein. In some embodiments, “about” refers to a range of values±10% of a specified value.

As used herein, the term “pharmaceutical” is used adjectivally to mean that the pharmaceutical combination is for use in a pharmaceutical product to prevent or treat diseases and complies with relevant regulatory requirements. When it is used, for example, to describe a specific component of the pharmaceutical combination, it characterizes the component as being compatible with the other ingredients of the composition and not disadvantageously deleterious to the intended goal.

The term “pharmaceutical combination” as used herein, refers to a combination or a pharmaceutical composition, for simultaneous, separate, or sequential use. A “combination” as used herein, defines especially a “kit of parts” in the sense that a tumor-homing bacteria and a dose of an immunostimulant can be dosed independently, either in separate form or by use of different fixed combinations. The individual components, i.e., the tumor-homing bacteria and the dose of an immunostimulant may be delivered simultaneously, chronologically staggered, or sequentially, with equal or differing durations separating the administration of each component. In some embodiments, the individual components, e.g., the tumor-homing bacteria and the dose of an immunostimulant, are formulated for simultaneous delivery in a fixed-dose combination (FDC) that includes the tumor-homing bacteria and the dose of an immunostimulant combined in a single dosage form, having a predetermined combination of respective dosages. Simultaneous delivery may reduce the risk of tolerance to bacteria.

As used herein, the term “cancer” refers to an uncontrolled, abnormal growth of a host's own cells which may lead to invasion of surrounding tissue and potentially tissue distal to the initial site of abnormal cell growth in the host. Major classes include carcinomas, which are cancers of the epithelial tissue (e.g., skin, squamous cells); sarcomas, which are cancers of the connective tissue (e.g., bone, cartilage, fat, muscle, blood vessels); leukemias, which are cancers of blood forming tissue (e.g., bone marrow tissue); lymphomas and myelomas, which are cancers of immune cells; and central nervous system cancers, which include cancers from brain and spinal tissue. “Cancer(s),” “neoplasm(s),” and “tumor(s)” are used herein interchangeably. As used herein, “cancer” refers to all types of cancer or neoplasm or malignant tumors including leukemias, carcinomas, and sarcomas, whether new or recurring. Treating cancer refers to inhibiting the growth or reducing the volume of a solid tumor cancer.

As used herein, the terms “subject,” “host,” or “patient” are used interchangeably to refer to a vertebrate such as a mammal. Mammals include but are not limited to primates and rodents. In some embodiments, the host is a human. Typically, the “subject,” “host,” or “patient” refers to a mammal, e.g., a human, having or susceptible to developing a tumor or cancer.

As used herein, “to treat” or “therapeutic,” and grammatically related terms, refer to any improvement of any consequence of disease, such as prolonged survival, less morbidity, and/or a lessening of side effects which are the byproducts of an alternative therapeutic modality. As is readily appreciated in the art, full eradication of disease is preferred but albeit not a requirement for a treatment act. “Treatment” or “treat,” as used herein, refers to the administration of a described pharmaceutical combination to a subject, e.g., a patient. The treatment can be to cure, heal, alleviate, relieve, alter, remedy, ameliorate, palliate, improve, or affect the disorder, or the symptoms of the disorder, e.g., a cancer.

As used herein, “physiologically acceptable carriers for parenteral administration” refers to any diluents, excipients, or solvents that may be used in the formulation of the pharmaceutical combination. Suitable pharmaceutical carriers are described in Remington's Pharmaceutical Sciences, Mack Publishing Company, a standard reference text in this field. They are selected with respect to the intended form of administration, that is, parenteral administration and the like, and are consistent with conventional pharmaceutical practices.

For the purpose of the present invention, genes are denoted by letters of lower case and italicized to be distinguished from proteins. In case a bacterial species name (like Salmonella typhimurium) is followed by a gene (denoted by letters of lower case and italicized), they refer to a mutation of the corresponding gene in the corresponding bacterial species. For example, stm3120 refers to the effector protein encoded by the stm3120 gene. Salmonella typhimurium stm3120 represents a S. typhimurium having a mutation in the stm3120 gene.

As used herein a “genetic modification” or “genetically modified” refers to a genetic engineering of a mutation and/or deletion and/or insertion of a gene, and can include genetic events that are directed in a laboratory setting and by design, e.g., through cell passaging and/or through one or more recombinant genetic methods. The term, “genetic attenuation” refers to any mutation or disruption of genes involved in the expression of a given protein and results in reduced or no expression of said one or more proteins. Ideally, the genetic attenuation(s) comprise gene deletions rather than point mutations to prevent spontaneous compensatory mutations that might result in reversion to a virulent phenotype.

In the context of a genetic modification, the phrase “genetically associated with” refers to any mechanism known in the art for associating control of a gene's expression genetically. For example, a gene may be genetically associated with a cis- or trans-regulatory element, including a cis-linked inducible promoter and a trans-regulatory element located in a distant genomic region or on a plasmid. Examples of regulatory elements are known in the art and include, e.g., promoters, transcription factors, enhancer or repressor molecules, and miRNA. As used herein, a “live bacteria” is viable bacteria.

As used herein, “engineered for tumor homing” means that a bacteria has been genetically modified to target a tumor. Such modifications may include any genetic modification such as upregulation or genetic attenuation, replacement or genetic attenuation, or insertion. The modifications may involve genetic transformation, transduction, or conjugation with a polynucleotide construct such as a vector.

The term “mutation” as used herein includes changes in both single and multiple base pairs. Such mutations may include substitutions, frame-shift mutations, deletions, insertions, and truncations.

The term “modulation” as used herein refers to both upregulation, such as activation or stimulation (e.g., by agonizing or potentiating) and downregulation, such as inhibition or suppression (e.g., by antagonizing, decreasing, or inhibiting).

The term “wild type strain” as used herein refers to a naturally occurring variant or a naturally occurring variant containing genetic modification in for example, antibiotic resistance genes.

“Constitutive toxicity in a host” refers to bacteria having constitutively active proteins involved in synthesis of an immunostimulant, or a derivative or precursor thereof, and/or constitutive expression of proteins involved in synthesis of an immunostimulant, or a derivative or precursor thereof.

The term “inducible” refers to gene expression that is not constitutive, but which takes place in response to a stimulus. The stimulus may be any suitable inducer known in the art, including but not limited to Vanillic acid, Isopropyl-D-1-thiogalactopyranoside (IPTG), Choline chloride (Cho), Naringenin, 3,4-Dihydroxybenzoic acid (DHBA), Sodium salicylate, 3-Hydroxytetradecanoyl-homoserine lactone (OHC14), Acrylic acid, Erythromycin, 2,4-Diacetylphophloroglucinol, Cuminic acid, or L-Arabinose.

As used herein, an “antigen” is any molecule that can be recognized by an immune response, either an antibody or by an immune cell (e.g., a T cell). As used herein, the terms “tumor-specific antigen” and “neoantigen” are used interchangeably and refer to an antigen that is expressed by a tumor but is not expressed by any normal cells in the organism from which the tumor was derived. The term “tumor-associated antigen” refers to an antigen that is expressed by a tumor but may also be expressed in a limited manner by normal cells in the organism from which the tumor was derived. The phrase “tumor antigen” encompasses “tumor-specific antigens,” neoantigens, and “tumor-associated antigens.”

As used herein, the terms “maximum tolerated dose” and “MTD” refer to the highest dose of a bacterial toxin that produces a toxic effect without causing life-threatening toxicity or mortality in the host. Suitable methods of calculating maximum tolerated dose (MTD) are known in the art. For example, MTD may be calculated by testing increasing doses in a subject, e.g., a human or a mouse, and establishing the dose prior to which unacceptable adverse effects occur. Unacceptable adverse effects may include more than 20% body weight loss in 10% of subjects or more than 15% weight loss in a single subject.

The pharmaceutical combinations described herein can be formulated using established techniques, such as microbial cultivation in fermenters, followed by processes like centrifugation for concentration and filtration or dialysis. Additional steps may include conventional granulation, mixing, dissolution, encapsulation, lyophilization, or emulsification. These formulations can take various forms such as granules, precipitates, particulates, powders (freeze-dried, rotary-dried, spray-dried), amorphous powders, injections, emulsions, elixirs, suspensions, or solutions.

The term “parenteral” as used herein includes subcutaneous, submucosal, intravenous, intratumoral, intra-peritoneal, intramuscular, intra-articular, intra-synovial, intrasternal, intrathecal, intrahepatic, intralesional, and intracranial injection, or infusion techniques. In some embodiments, the parenteral route is intratumoral, intra-peritoneal, or intravenous. In some embodiments, the parenteral route is intratumoral. In some embodiments, the parenteral route is intra-peritoneal. In some embodiments, the parenteral route is intravenous.

As used herein, the “immunoactive-transient-bacteria” is a live bacteria genetically modified for transient biosynthesis or down-regulation of an immunostimulant. The “immunoactive-transient-bacteria” exhibits transiently-activating toxicity in a host by any acceptable mechanism. This may include but is not limited to transient expression of an immunostimulant or clearance of the bacteria from a host after a specified timeframe.

A “bacterial adjuvant” is is any whole bacteria or portion thereof sufficient to stimulate an immune response in a host, and includes live intact bacteria, inactivated intact bacteria (ghosts), and outer membrane vesicles (OMV), but does not include isolated PAMPs such as LPS molecules.

The term “leaky promoter” refers to a gene control element that never achieves 100% shut-off (e.g., when no inducer is present for an inducible promoter or when a repressor molecule is present), thereby allowing for a small but detectable amount of basal transcription (gene expression) measured by mRNA or protein levels. The term “leaky expression” of a gene refers to the low, but detectable, basal level of gene expression that occurs under a leaky promoter and can be assessed by detectable mRNA or protein levels when transcription is turned “off”, e.g., because no inducer is present for an inducible promoter or because a repressor molecule is present.

Unless otherwise indicated, the nucleic acid sequences of genes disclosed are available to those skilled in the art, e.g., in the GenBank Database.

The present disclosure provides pharmaceutical combinations and methods for maximizing efficacy of a tumor-homing bacteria while exhibiting desirably low levels of toxicity in a host. The disclosed pharmaceutical combinations thus address two distinct and contradictory strategies for host immune system activation and human serum survival. The pharmaceutical combinations and methods disclosed herein exhibit desirable human serum survival and/or tumor-homing when the tumor-homing bacteria is administered at low doses. In some embodiments, the tumor-homing bacteria comprises toxicity reduction of an immunostimulant. The pharmaceutical combination and methods disclosed herein further comprise a host-transient immunostimulant. In some embodiments, the host-transient immunostimulant induces host immune system activation sufficient to enhance the efficacy of the tumor-homing bacteria.

In some embodiments, the disclosed pharmaceutical combinations comprise a dose of tumor-homing bacteria that is at least 50% less than the tumor-homing bacteria's MTD, and a host-transient bacterial adjuvant comprising a supplementary dose of an immunostimulant, or a derivative or precursor thereof. In some embodiments, the dose of tumor-homing bacteria is at least 60% less than the tumor-homing bacteria's MTD. In some embodiments, the dose of tumor-homing bacteria is at least 70% less than the tumor-homing bacteria's MTD. In some embodiments, the dose of tumor-homing bacteria is at least 80% less than the tumor-homing bacteria's MTD. In some embodiments, the dose of tumor-homing bacteria is at least 90% less than the tumor-homing bacteria's MTD. In some embodiments, the dose of tumor-homing bacteria is at least 95% less than the tumor-homing bacteria's MTD.

In some embodiments, the disclosed pharmaceutical combinations comprise one or more physiologically acceptable carriers and two bacterial strains. In some embodiments, the two bacterial strains are an immunoactive-transient-bacteria genetically modified for transient survival in human serum, inducible synthesis of an immunostimulant, or transient synthesis of an immunostimulant; and a tumor-homing bacteria which is an immunoactive-constitutive-bacteria. The tumor-homing bacteria may act as an immunostimulant whether administered in wild-type form with respect to the immunostimulant or when modified for reduced toxicity with respect to the immunostimulant. The disclosed pharmaceutical combinations surprisingly achieve desirable efficacy, including a reduction in mouse mortality, a notable decrease in bacteremia at an extended post-administration time point, and minimal additional toxicity as measured by weight recovery.

In some embodiments, the pharmaceutical combination is configured to exhibit a substantial increase in endotoxin and/or pyrogenic activity in a host at the time of administration, followed by a reduction to minimal or basal endotoxin and/or pyrogenic activity in the host at least 6 hours post administration. In some embodiments, the reduction in endotoxin and/or pyrogenic activity in the host is measured at least 8 hours post administration. In some embodiments, the reduction in endotoxin and/or pyrogenic activity in the host is measured at least 12 hours post administration. In some embodiments, the reduction in endotoxin and/or pyrogenic activity in the host is measured at least 16 hours post administration. In some embodiments, the reduction in endotoxin and/or pyrogenic activity in the host is measured at least 20 hours post administration. In some embodiments, the reduction in endotoxin and/or pyrogenic activity in the host is measured at least 24 hours post administration. In some embodiments, the reduction in endotoxin and/or pyrogenic activity in the host is measured at least 36 hours post administration. In some embodiments, the reduction in endotoxin and/or pyrogenic activity in the host is measured at least 48 hours post administration. In some embodiments, the reduction in endotoxin and/or pyrogenic activity in the host is measured at least 72 hours post administration. The reduction to a minimal endotoxin and/or pyrogenic activity in the host may encompass a change from acute to chronic activity.

Further provided herein is a pharmaceutical combination comprising a tumor-homing bacteria, a host-transient bacterial adjuvant comprising a supplementary dose of an immunostimulant, or a derivative or precursor thereof, and one or more physiologically acceptable carriers for parenteral administration. The immunostimulant, derivative, or precursor thereof may act as an endotoxic adjunct or adjuvant to the tumor-homing bacteria. In some embodiments, the adjunct or adjuvant is distinct from and not physically connected to the tumor-homing bacteria.

In some embodiments, the host-transient bacterial adjuvant comprising a supplementary dose is configured for significant reduction of the immunostimulant, derivative, or precursor thereof, in the blood stream of a host at least 6 hours post administration. In some embodiments, the reduction of the immunostimulant in the host's bloodstream is measured at least 8 hours post administration. In some embodiments, the reduction of the immunostimulant in the host's bloodstream is measured at least 12 hours post administration. In some embodiments, the reduction of the immunostimulant in the host's bloodstream is measured at least 16 hours post administration. In some embodiments, the reduction of the immunostimulant in the host's bloodstream measured at least 20 hours post administration. In some embodiments, the reduction of the immunostimulant in the host's bloodstream is measured at least 24 hours post administration.

The host-transient bacterial adjuvant comprising the supplementary dose of the immunostimulant, derivative, or precursor thereof, may be in the form of a molecule, portion or derivative of a microbe, killed microbe, or live microbe.

Also provided herein is a pharmaceutical combination comprising an immunoactive-transient-bacteria genetically modified for transient survival in human serum, inducible synthesis of an immunostimulant, or transient synthesis of an immunostimulant, and a tumor-homing bacteria which is an immunoactive-constitutive-bacteria. In some embodiments, the immunoactive-constitutive-bacteria comprises an immunostimulant. One or both of the bacteria may be genetically modified for reduced toxicity of the immunostimulant. In some embodiments, the immunoactive-transient-bacteria is genetically modified with two types of genetic modifications with respect to the immunostimulant—a constitutively functional first attenuation which is capable of reducing potency or toxicity of the immunostimulant, and transiently active or inducible synthesis of an immunostimulant. In some embodiments, the immunostimulant comprises a pathogen-associated molecular pattern (PAMP).

Methods of using the disclosed pharmaceutical combinations to induce an immune response and/or to enhance tumor-homing in a host are also provided herein. Methods are also provided herein for using the disclosed pharmaceutical combinations to prevent, delay progression of, or treat a cancer.

Further provided is a method of modulating an immune system response and enhancing tumor colonization in a host with cancer, comprising administering to a host a tumor-homing bacteria and a host-transient bacterial adjuvant comprising a supplementary dose of an immunostimulant, or a derivative or precursor thereof. In some embodiments, the administration of the bacteria and/or bacterial adjuvant is by parenteral administration. In some embodiments, the supplementary dose of the immunostimulant, derivative, or precursor, is configured for significant reduction in the blood stream of a host at least 6 hours post administration. In some embodiments, the supplementary dose of the immunostimulant, derivative, or precursor, is configured for significant reduction in the blood stream of a host at least 12 hours post administration. In some embodiments, the supplementary dose of the immunostimulant, derivative, or precursor, is configured for significant reduction in the blood stream of a host at least 12 hours post administration. In some embodiments, the supplementary dose of the immunostimulant, derivative, or precursor, is configured for significant reduction in the blood stream of a host at least 24 hours post administration. In some embodiments, the supplementary dose of the immunostimulant, derivative, or precursor, is configured for significant reduction in the blood stream of a host at least two days post administration. In some embodiments, the supplementary dose of the immunostimulant, derivative, or precursor, is configured for significant reduction in the blood stream of a host at least five days post administration. In some embodiments, the supplementary dose of the immunostimulant, derivative, or precursor, is configured for significant reduction in the blood stream of a host at least seven days post administration.

Also provided is a method of modulating an immune system response and enhancing tumor colonization in a host with cancer, comprising parenteral administration of an immunoactive-inducible strain and a toxicity-reduced immunoactive-constitutive-bacteria.

Further provided herein are therapeutic uses of the disclosed pharmaceutical combinations and pharmaceutical combinations for use in modulating an immune system response and enhancing tumor-homing in a subject; and/or preventing, delaying the progression of, or treating a cancer.

Another exemplary embodiment is the use of the pharmaceutical combinations disclosed herein in the manufacture of a medicament for modulating an immune system response and enhancing tumor-homing in a subject; and/or preventing, delaying the progression of, or treating a cancer.

Compositions described herein may be formulated in a variety of ways for use in the methods described herein. In one embodiment, the composition as described throughout comprises the bacteria and a one or more physiologically acceptable carriers.

### Pharmaceutical Combinations

Provided herein is a pharmaceutical combination, and methods of using the pharmaceutical combination in modulation of a host immune system and enhanced tumor homing. In some embodiments, the pharmaceutical combination exhibits improved human serum resilience for tumor-homing. In some embodiments, the present disclosure provides a pharmaceutical combination comprising: a dose of tumor-homing bacteria and an immunostimulant, or a derivative or precursor thereof. In some embodiments, the pharmaceutical combination further comprises one or more physiologically acceptable carriers for parenteral administration. In some embodiments, the derivative of the immunostimulant is a toxicity-reduced derivative of the immunostimulant.

In some embodiments, the immunostimulant comprises LPS.

In some embodiments, the pharmaceutical combination comprises a dose of tumor-homing bacteria and a host-transient bacterial adjuvant. In some embodiments, the host-transient bacterial adjuvant comprises a supplementary dose of an immunostimulant, or a derivative or precursor thereof. In some embodiments, the pharmaceutical combination further comprises one or more physiologically acceptable carriers for parenteral administration. In some embodiments, the derivative of the immunostimulant is a toxicity-reduced derivative of the immunostimulant.

In some embodiments, the dose of tumor-homing bacteria is a dose that is at least 50% less than the tumor-homing bacteria's MTD. In some embodiments, the dose of tumor-homing bacteria is a dose that is at least 60% less than the tumor-homing bacteria's MTD. In some embodiments, the dose of tumor-homing bacteria is a dose that is at least 70% less than the tumor-homing bacteria's MTD. In some embodiments, the dose of tumor-homing bacteria is a dose that is at least 80% less than the tumor-homing bacteria's MTD. In some embodiments, the dose of tumor-homing bacteria is a dose that is at least 90% less than the tumor-homing bacteria's MTD. In some embodiments, the dose of tumor-homing bacteria is a dose that is at least 95% less than the tumor-homing bacteria's MTD. In some embodiments, the dose of tumor-homing bacteria is a dose that is at least 99% less than the tumor-homing bacteria's MTD.

Further provided is a pharmaceutical combination and uses thereof, comprising a dose of tumor-homing bacteria, a host-transient bacterial adjuvant comprising a supplementary dose of an immunostimulant, or a derivative or precursor thereof, and one or more physiologically acceptable carriers for parenteral administration. In some embodiments, the derivative of the immunostimulant is a toxicity-reduced derivative of the immunostimulant.

There is also provided a pharmaceutical combination formulated as a single composition comprising one or more physiologically acceptable carriers and two bacterial strains. In some embodiments, the bacterial strains comprise an immunoactive-transient-bacteria and a tumor-homing bacteria which is an immunoactive-constitutive-bacteria. In some embodiments, the immunoactive-transient-bacteria is genetically modified for transient survival in human serum, inducible synthesis of the immunostimulant, or transient synthesis of the immunostimulant. In some embodiments, the immunoactive-constitutive-bacteria constitutively produces an immunostimulant. In some embodiments, the pharmaceutical combination further comprises one or more physiologically acceptable carriers. In some embodiments, one or both bacteria are genetically modified for reduced toxicity of the immunostimulant. In some embodiments, both bacterial strains are genetically modified for reduced toxicity of the immunostimulant. For example, the tumor-homing bacteria and/or the immunoactive-transient-bacteria may be genetically modified for reduced toxicity of the immunostimulant. In some embodiments, the immunostimulant, derivative, or precursor comprises Lipid A. In some embodiments, the immunostimulant, derivative, or precursor comprises LPS.

In various embodiments, the immunoactive-transient-bacteria is an immunoactive inducible strain and the pharmaceutical combination further comprises an inducer. In some embodiments, the immunoactive-transient-bacteria is an immunoactive inducible strain and the pharmaceutical combination includes an insignificant amount of an inducer, such as trace amounts of an inducer or an undetectable amount of an inducer. For example, an inducer may have been present in the pharmaceutical combination during the manufacturing stages, but was removed prior to providing a final pharmaceutical combination for administration to a host. Methods of measuring inducer molecules are known in the art and include, e.g., high-performance liquid chromatography (HPLC). In some embodiments, one or both of the immunoactive-constitutive-bacteria and the immunoactive-transient-bacteria are genetically modified for reduced toxicity of the immunostimulant.

As used herein, genetically modified for reduced toxicity refers to a genetic modification which provides attenuated activity in a host of Toll-like receptor 4 (TLR4), Toll-like receptor 3 (TLR3), Toll-like receptor 1 (TLR1), Toll-like receptor 7 (TLR7), Toll-like receptor 5 (TLR5), or Toll-like receptor 2 (TLR2); reduced TNF-alpha secretion or IFN-gamma secretion; and/or complement activation, compared to activity in the host of a bacteria without the genetic modification. In some embodiments, genetically modified for reduced toxicity refers to a genetic modification which provides attenuated activity in a host of TLR4 or TLR5. In some embodiments, the genetic modification provides attenuated activity in a host of TLR4. In some embodiments, the genetic modification provides attenuated activity in a host of TLR5.

In some embodiments, the immunoactive-transient-bacteria is an immunoactive inducible strain and the pharmaceutical combination comprises an inducer. In some embodiments, the inducer is present in trace amounts. In some embodiments, when exposed to the inducer, the immunoactive-transient-bacteria synthesizes the immunostimulant, derivative, or precursor. In some embodiments, the immunoactive-transient-bacteria gradually loses the ability to synthesize the immunostimulant, derivative, or precursor when the inducer is no longer present. Note that the induction is with respect to a specific immunostimulant. In some embodiments, the immunoactive-transient-bacteria comprises flagella. In some embodiments, the flagella are functional in the bacteria's induced state and not functional or not substantially present in the bacteria's uninduced state. In some embodiments, the immunostimulant comprises LPS. In some embodiments, the immunostimulant comprises Lipid A. In some embodiments, the LPS and/or the Lipid A is functional in the bacteria's induced state and not functional or not substantially present in the bacteria's uninduced state. In some embodiments, the tumor-homing bacteria exhibits constitutive toxicity in a host. In some embodiments, the tumor-homing bacteria exhibits human serum resilience at 3 hours after parenteral administration of more than 3-fold relative to S. typhimurium VNP20009. Constitutive toxicity in a host refers to bacteria having constitutively expression of active proteins involved in synthesis of an immunostimulant, or a derivative or precursor thereof.

In some embodiments, the immunoactive-constitutive-bacteria has a percent viability in human serum at 3 hours of more than 5-fold. Percent viability is determined by assessing the viability of 10⁵ colony forming units (CFU) of bacteria after exposure to human serum at 37° C. for 3 hours compared to the total CFU surviving when incubated in heat inactivated serum at 37° C.

In some embodiments, the immunoactive-constitutive-bacteria has a percent viability in human serum at 3 hours of more than 8-fold relative to S. typhimurium VNP20009. In some embodiments, the immunoactive-constitutive-bacteria has a percent viability in human serum at 3 hours of more than 10-fold relative to S. typhimurium VNP20009. In some embodiments, the immunoactive-constitutive-bacteria has a percent viability in human serum at 3 hours of more than 12-fold relative to S. typhimurium VNP20009. In some embodiments, the immunoactive-constitutive-bacteria has a percent viability in human serum at 3 hours of more than 15-fold relative to S. typhimurium VNP20009. In some embodiments, the immunoactive-constitutive-bacteria has a percent viability in human serum at 3 hours of more than 20-fold relative to S. typhimurium VNP20009. In some embodiments, the immunoactive-constitutive-bacteria has a percent viability in human serum at 3 hours of more than 25-fold relative to S. typhimurium VNP20009. In some embodiments, the immunoactive-constitutive-bacteria has a percent viability in human serum at 3 hours of more than 30-fold relative to S. typhimurium VNP20009.

In some embodiments, the dose of the tumor-homing bacteria is substantially less than a MTD for a given tumor-homing bacteria. In some embodiments, the given tumor-homing bacteria for determining the MTD is S. typhimurium VNP20009. “Substantially less” than a MTD for a given tumor-homing bacteria may comprise a dose of the tumor-homing bacteria in the pharmaceutical composition of less than 30% of the MTD of the given tumor-homing bacteria. In some embodiments, the dose of tumor-homing bacteria is at least 30% less than the MTD of the given tumor-homing bacteria. In some embodiments, the dose of tumor-homing bacteria is at least 20% less than the MTD of the given tumor-homing bacteria. In some embodiments, the dose of tumor-homing bacteria is at least 10% less than the MTD of the given tumor-homing bacteria. In some embodiments, the dose of tumor-homing bacteria is at least 5% less than the MTD of the given tumor-homing bacteria. In some embodiments, the dose of tumor-homing bacteria is at least 3% less than the MTD of the given tumor-homing bacteria. In some embodiments, the dose of tumor-homing bacteria is at least 1% less than the MTD of the given tumor-homing bacteria. In some embodiments, the dose of tumor-homing bacteria is at least 0.5% less than the MTD of the given tumor-homing bacteria. In some embodiments, the dose of tumor-homing bacteria is at least 0.05% less than the MTD of the given tumor-homing bacteria.

In some embodiments, the dose of tumor-homing bacteria is at least 30% less than a MTD of S. typhimurium VNP20009. In some embodiments, the dose of tumor-homing bacteria is at least 20% less than a MTD of S. typhimurium VNP20009. In some embodiments, the dose of tumor-homing bacteria is at least 10% less than a MTD of S. typhimurium VNP20009. In some embodiments, the dose of tumor-homing bacteria is at least 5% less than a MTD of S. typhimurium VNP20009. In some embodiments, the dose of tumor-homing bacteria is at least 3% less than a MTD of S. typhimurium VNP20009. In some embodiments, the dose of tumor-homing bacteria is at least 1% less than a MTD of S. typhimurium VNP20009. In some embodiments, the dose of tumor-homing bacteria is at least 0.5% less than a MTD of S. typhimurium VNP20009. In some embodiments, the dose of tumor-homing bacteria is at least 0.05% less than a MTD of S. typhimurium VNP20009.

In some embodiments, the dose of tumor-homing bacteria is less than 10⁸ CFU/m². In some embodiments, the dose of tumor-homing bacteria is less than 5×10⁷ CFU/m². In some embodiments, the dose of tumor-homing bacteria is less than 10⁷ CFU/m². In some embodiments, the dose of tumor-homing bacteria is less than 5×10⁶ CFU/m². In some embodiments, the dose of tumor-homing bacteria is less than 10⁶ CFU/m². In some embodiments, the dose of tumor-homing bacteria is less than 5×10⁵ CFU/m².

In some embodiments, the dose of tumor-homing bacteria is from 10⁴ to 10⁷, 10⁴ to 5×10⁶, 10⁴ to 10⁶, 10⁴ to 5×10⁶, 10⁴ to 10⁶, 10⁴ to 5×10⁵, 10⁴ to 10⁵ CFU/m². In some embodiments, the dose of tumor-homing bacteria is from 10⁵ to 10⁷, 10⁵ to 5×10⁶, 10⁵ to 10⁶, 10⁵ to 5×10⁶, or 10⁵ to 10⁶ CFU/m². Each possible value within the recited ranges represents a separate embodiment of the present invention.

In some embodiments, the dose of tumor-homing bacteria is less than a maximum tolerated dose of S. typhimurium VNP20009. In some embodiments, less than the maximum tolerated dose of S. typhimurium VNP20009 comprises a dose of tumor-homing bacteria of less than 10⁷ CFU/m². In some embodiments, less than the maximum tolerated dose of S. typhimurium VNP20009 comprises a dose of tumor-homing bacteria of less than 10⁶ CFU/m². In some embodiments, the tumor-homing bacteria is oncolytic.

In some embodiments, the tumor-homing bacteria comprises flagella. In some embodiments, the flagella are intact.

In some embodiments, the pharmaceutical combination is a kit. In some embodiments, the pharmaceutical combination is a single formulation. In some embodiments, the tumor-homing bacteria and the host-transient bacterial adjuvant are in separate compositions. In some embodiments, the tumor-homing bacteria and the immunoactive-transient-bacteria are in separate compositions.

In some embodiments of the host-transient bacterial adjuvant comprising the supplementary dose of the immunostimulant, derivative, or precursor, the supplementary dose is at least 15% of the MTD of the immunostimulant, derivative, or precursor thereof. In some embodiments, the supplementary dose is at least 20% of the MTD of the immunostimulant, derivative, or precursor thereof. In some embodiments, the supplementary dose is at least 25% of the MTD of the immunostimulant, derivative, or precursor thereof. In some embodiments, the supplementary dose is at least 30% of the MTD of the immunostimulant, derivative, or precursor thereof. In some embodiments, the supplementary dose is at least 40% of the MTD of the immunostimulant, derivative, or precursor thereof. In some embodiments, the supplementary dose is at least 50% of the MTD of the immunostimulant, derivative, or precursor thereof. In some embodiments, the supplementary dose is at least 60% of the MTD of the immunostimulant, derivative, or precursor thereof. In some embodiments, the supplementary dose is at least 10% of the MTD of the immunostimulant, derivative, or precursor thereof.

In some embodiments of the host-transient bacterial adjuvant comprising a supplementary dose of the immunostimulant, derivative, or precursor, the supplementary dose of the immunostimulant is sufficient to provide a toxicity (e.g., an endotoxicity) increase of at least 50%, at least 70%, at least 75%, at least 80%, at least 85%, at least 90%, or at least 95% relative to the toxicity of the dose of the tumor-homing bacteria.

In some embodiments, the host-transient bacterial adjuvant or immunoactive-transient-bacteria is sufficient to provide a toxicity (e.g., an endotoxicity) increase of at least 30%, at least 50%, at least 70%, at least 75%, at least 80%, at least 85%, at least 90%, or at least 95% relative to the dose of the tumor-homing bacteria. In some embodiments, the host-transient bacterial adjuvant or immunoactive-transient-bacteria is sufficient to provide a toxicity (e.g., an endotoxicity) increase of at least 30% relative to the dose of the tumor-homing bacteria. In some embodiments, the host-transient bacterial adjuvant or immunoactive-transient-bacteria is sufficient to provide a toxicity (e.g., an endotoxicity) increase of at least 50%, relative to the dose of the tumor-homing bacteria. In some embodiments, the host-transient bacterial adjuvant or immunoactive-transient-bacteria is sufficient to provide a toxicity (e.g., an endotoxicity) increase of at least 70% relative to the dose of the tumor-homing bacteria. In some embodiments, the host-transient bacterial adjuvant or immunoactive-transient-bacteria is sufficient to provide a toxicity (e.g., an endotoxicity) increase of at least 75% relative to the dose of the tumor-homing bacteria. In some embodiments, the host-transient bacterial adjuvant or immunoactive-transient-bacteria is sufficient to provide a toxicity (e.g., an endotoxicity) increase of at least 80% relative to the dose of the tumor-homing bacteria. In some embodiments, the host-transient bacterial adjuvant or immunoactive-transient-bacteria is sufficient to provide a toxicity (e.g., an endotoxicity) increase of at least 85% relative to the dose of the tumor-homing bacteria. In some embodiments, the host-transient bacterial adjuvant or immunoactive-transient-bacteria is sufficient to provide a toxicity (e.g., an endotoxicity) increase of at least 90% relative to the dose of the tumor-homing bacteria. In some embodiments, the host-transient bacterial adjuvant or immunoactive-transient-bacteria is sufficient to provide a toxicity (e.g., an endotoxicity) increase of at least 95% relative to the dose of the tumor-homing bacteria.

In some embodiments, the dose of tumor-homing bacteria is at least 50% less than the tumor-homing bacteria's maximum tolerated dose, and the dose of bacterial adjuvant or immunoactive-transient-bacteria is sufficient to provide a toxicity increase of at least 30% relative to the toxicity of the dose of the tumor-homing bacteria.

In some embodiments, the host-transient bacterial adjuvant exhibits a transient toxicity in a host blood stream as measured by a return of a toxicity measure to basal levels (i.e., +/−20% or +/−10% of basal levels) in a host body at least 6 hours post administration. In some embodiments, the host-transient bacterial adjuvant exhibits a transient toxicity in a host blood stream as measured by a return of a toxicity measure to basal levels in a host body at least 12 hours post administration. In some embodiments, the host-transient bacterial adjuvant exhibits a transient toxicity in a host blood stream as measured by a return of a toxicity measure to basal levels in a host body at least 24 hours post administration. In some embodiments, the host-transient bacterial adjuvant exhibits a transient toxicity in a host blood stream as measured by a return of a toxicity measure to basal levels in a host body at least two days post administration. In some embodiments, the host-transient bacterial adjuvant exhibits a transient toxicity in a host blood stream as measured by a return of a toxicity measure to basal levels in a host body at least five days post administration. In some embodiments, the host-transient bacterial adjuvant exhibits a transient toxicity in a host blood stream as measured by a return of a toxicity measure to basal levels in a host body at least seven days post administration. As will be recognized by those skilled in the art, there are many examples of suitable toxicity measures, including but not limited to IL6 levels, weight loss, weight recovery, fever, and bacteremia. For example, in the case of a toxicity measure being weight loss, within 7 days post administration, the weight of the host substantially returns to basal levels.

In some embodiments, the pharmaceutical combination does not exhibit significantly greater toxicity in a host than the toxicity caused by any one of the components (i.e., the tumor-homing bacteria or bacterial adjuvant) when administered alone. In this context, not significantly greater toxicity refers to not more than 20% or not more than 10% increase in a quantitative measure used to evaluate toxicity, such as weight loss or weight recovery.

In some embodiments, the host-transient bacterial adjuvant comprising a supplementary dose of the immunostimulant, derivative, or precursor, is in an amount such that a total dose at administration of the immunostimulant, derivative, or precursor, including that provided by the tumor-homing bacteria, is substantially similar to a maximum tolerated dose of the immunostimulant.

In some embodiments, the supplementary dose of the immunostimulant, derivative, or precursor, is substantially similar to a maximum tolerated dose for a given immunostimulant.

In some embodiments, the host-transient bacterial adjuvant is chosen from: a synthetic immunostimulant, a bacteria-derived particle comprising an immunostimulant (e.g., outer membrane vesicles), an inactivated bacteria comprising an immunostimulant (also referred to as a killed bacteria or “ghost bacteria”), a live bacteria comprising an immunostimulant configured to be transiently present in the blood stream of a host, and an immunoactive-transient-bacteria. In some embodiments, the bacterial adjuvant comprises a live bacteria comprising an immunostimulant configured to be transiently present in the blood stream of a host. In some embodiments, the bacterial adjuvant comprises an immunoactive-transient-bacteria. In some embodiments, the live bacteria comprising an immunostimulant configured to be transiently present in the blood stream of a host is a commensal bacteria or a bacteria with reduced pathogenicity.

Synthetic immunostimulants are known in the art and include but are not limited to a polyinosinic: polycytidylic acid which binds TLR3; Pam2CSK4 (Pam2), a diacylated lipopeptide which binds and activates the TLR2 and TLR6 pathways; Pam3CSK4 (Pam3), which binds and activates the TLR2 and TLR1 pathways; R848 and R837, which are imidazoquinolines that bind and activate the TLR7 and TLR8 pathways; and unmethylated CpG motif sequences (oligodeoxynucleotides (ODNs)), which are common to certain bacteria and bind to and activate TLR9.

In some embodiments, the synthetic immunostimulant is a toxicity reduced immunostimulant in respect to “wild-type” immunostimulant. In some embodiments, the synthetic immunostimulant is or comprises lipid A, altered LPS structures with truncated or modified O-antigens, or modified Lipid A. Examples of modifications to lipid A are known in the art and include monophosphorylated lipid A (MPLA or MPL), which has a reduced ability to activate TLR4. Alternative examples include liposome-based combinations of MPL such as liposome-based combinations or oil-in-water emulsions of MPL and saponin. Examples of such synthetic lipid A analogues include RC-529, E6020, glucopyranosyl lipid A (GLA), ONO-4007, E5564, and CRX-526. The synthetic immunostimulant may be a bacterial or microbial product.

In some embodiments, the host-transient bacterial adjuvant comprising the supplementary dose of the immunostimulant, derivative, or precursor, is configured to exhibit transient presence in a host blood stream (i.e., reduced presence in the blood at least 6 hours post administration.

In some embodiments, the host-transient bacterial adjuvant comprises a genetically modified bacteria configured to exhibit reduction in the blood stream of a host at least 6 hours post administration. In some embodiments, configured to exhibit reduction refers to a bacterial strain genetically modified for control of synthesis. For example, control of synthesis may be achieved by treatment with an exogenous agent. In some embodiments, configured to exhibit reduction refers to a bacterial strain genetically modified to comprise a mutation that results in an inability of the bacteria to survive in a mammalian host or in human serum. Configured to exhibit reduction in the blood stream of a host at least 6 hours post administration may refer to a genetic modification to halt synthesis of the immunostimulant, either prior to administration or shortly after administration. Such a genetic modification may provide for a controlled induction of the immune system with toxicity levels which decrease dramatically at least 6 hours post administration. In some embodiments, bacteria adapted for significant reduction in a blood stream are strains that have their synthesis reduced by at least 70%, at least 80%, at least 85%, at least 90%, at least 95%, at least 99%, or more, compared to an unmodified bacterium. In some embodiments of bacteria that do not reduce synthesis or reduce presence of the immunostimulant (e.g., LPS) completely, the bacteria are further treated or modified such that they cannot proliferate within a mammalian host.

In some embodiments, the pharmaceutical combination provides for a total dose of immunostimulant at administration which is substantially similar to a maximum tolerated dose. In some embodiments, the pharmaceutical combination is formulated as a kit, is present in two compositions, or is present as a single formulation for simultaneous administration.

In some embodiments, the host-transient bacterial adjuvant comprising the supplementary dose of the immunostimulant, derivative, or precursor, comprises a genetically engineered bacteria having variable survival in human serum, and/or transient biosynthesis of the immunostimulant, derivative, or precursor. In some embodiments, the genetically engineered (i.e., modified) bacteria is configured for reduction of its levels in the blood stream of a host at least 6 hours post administration, while the tumor-homing bacteria exhibits constitutive synthesis of an immunostimulant, derivative, or precursor.

In some embodiments, the dose of the immunoactive-transient-bacteria is significantly greater than the dose of the tumor-homing bacteria.

In some embodiments, the immunostimulant comprises PAMPs associated with bacteria. Exemplary PAMPs include factors such as bacterial lipopolysaccharide (LPS), bacterial flagellin, or viral double stranded RNA (dsRNA).

The TLRs are a class of pattern recognition receptors (PRRs), which detect pathogenic molecules, e.g., PAMPs. The TLR immune receptors are expressed on the membranes of leukocytes, including dendritic cells, and the binding of TLR agonists triggers molecular events that can lead to an immune response and antigen-specific acquired immunity. “Toll-like receptor 5 (TLR5) agonist” refers to a molecule that provides for TLR5-mediated inflammation. Examples include flagellin. Bacterial LPS is a strong agonist of TLR4 with the ability to enhance immune responses to soluble antigens. LPS stimulates immune system cells via the TLR4 pathway, which recognizes common PAMPs.

TLR4 is a transmembrane protein that, in conjunction with MD-2 and CD14 co-receptors, forms a crucial mammalian LPS detection system. Located on the external membrane of Gram-negative bacteria and some Gram-positive bacteria such as Negativicutes, TLR4 activation triggers signaling cascades involving NF-κB and TRIF. This leads to the production of proinflammatory cytokines such as IL-1, TNFα, IFNγ, and IL-12, along with co-stimulatory molecules that activate innate immunity by maturing dendritic cells, macrophages, and B cells. Additionally, it primes the adaptive immune response by enhancing the function of T and B cells.

LPS is a glycophospholipid connected through covalent bonding and consists of three fundamental components: 1) O antigen, a repetitive glycan polymer attached to the core oligosaccharide, forming the outermost segment of the molecule; 2) Core oligosaccharide, which is directly connected to lipid A and typically contains heptose and 3-deoxy-D-mannooctulosonic acid (KDO), also known as keto-deoxyoctulosonate; and 3) Lipid A, a phosphorylated glucosamine disaccharide linked to multiple fatty acids, which anchors LPS into the bacterial membrane while allowing the remainder of the molecule to protrude from the cell surface.

LPS is a glycophospholipid consisting of an antigenic, variable-size, carbohydrate chain covalently linked to lipid A, a conserved hydrophobic region. Toxicity of LPS is mediated by lipid A through its interaction with B-cells and macrophages of the mammalian immune system, a process leading to the secretion of proinflammatory cytokines, including TNFα, which may have fatal consequences for the host.

In some embodiments, the immunostimulant is a TLR2 agonist, a TLR4 agonist (e.g., LPS), a TLR5 agonist (e.g., Flagellin), a TLR3 agonist, a TLR1 agonist, a TLR7 agonist, a complement activator, or a TNF-alpha and/or IFN-gamma secretion inducer. In some embodiments, the immunostimulant is a TLR2 agonist, a TLR4 agonist, a TLR5 agonist, a complement activator, or a TNF-alpha and/or IFN-gamma secretion inducer. In some embodiments, the immunostimulant is a TLR4 agonist or a TLR5 agonist. The immunostimulant may be a cell surface protein that induces IL-6 secretion from host immune cells. “Agonist” refers to a substance that produces a biological response, typically by binding and activation of a receptor in a cell. In some embodiments, the immunostimulant is a TLR4 agonist.

In some embodiments, the immunoactive-transient-bacteria is genetically modified for transient synthesis of an immunostimulant. In some embodiments, the genetic modification comprises a deletion or attenuation of a gene encoding a protein involved in synthesis of the immunostimulant. In some embodiments, the genetic modification comprises insertion of a gene encoding a protein involved in synthesis of the immunostimulant genetically associated with a mechanism for transient synthesis of the immunostimulant.

Mechanisms for transient synthesis are known in the art and include associating a protein involved in synthesis of the immunostimulant with an inducible system. In some embodiments, the immunoactive-transient-bacteria is an immunoactive inducible bacteria and the pharmaceutical combination is a composition without the presence of an appropriate inducer. In some embodiments, the immunoactive-transient-bacteria is genetically modified for inducible synthesis of the immunostimulant. In some embodiments, the mechanism for transient synthesis of the immunostimulant is an inducible prokaryotic promoter. In some embodiments, the mechanism for transient synthesis comprises leaky expression of the immunostimulant when not exposed to an inducer. In some embodiments, leaky expression in an uninduced state results in low levels of synthesis of the immunostimulant when the inducer is less than minimally present or not detectable in the formulation. An acceptable inducible system may be selected based on an acceptable toxicity despite leaky expression of the immunostimulant during the uninduced state. In some embodiments, rfaD synthesis occurs at a low level in an inducible system when not exposed to an inducer, wherein the low level of rfaD expression maintains acceptable toxicity.

In some embodiments, the immunoactive-transient-bacteria is an immunoactive inducible bacterial strain and the pharmaceutical combination does not comprise an inducer or comprises trace amounts of an inducer.

In some embodiments, the tumor-homing bacteria is genetically modified for reduced toxicity of an immunostimulant. In some embodiments, the genetic modification for reduced toxicity of the immunostimulant comprises a genetic modification of a gene related to biosynthesis of the immunostimulant. The biosynthesis may involve a post-synthesis toxicity reduction by structural modification of the immunostimulant compared to a bacteria without the genetic modification. The immunostimulant may be a cell surface protein. Genetic modification for reduced toxicity may refer to a modification, deletion, or insertion of a gene selected from: pagE, pagP, pagL, lpxM, lpxL, lpxE, lpxR, lpxF, lpxXL, and lpxT. A structural modification may be provided by modification, deletion, or insertion of a gene selected from: lpxXL, lpxF, and lpxE. In some embodiments, the genetic modification is a modification, deletion, and/or insertion of lpxE.

In some embodiments, the immunostimulant is Lipid A, LPS, flagellin, lipoprotein, fimbriae, peptidoglycan, lipoteichoic (LTA), core oligosaccharide, or O-antigen. In some embodiments, the immunostimulant is Lipid A or LPS.

In some embodiments, at least one bacteria in the pharmaceutical combination is a Gram-negative bacteria. In some embodiments, the tumor-homing bacteria is a Gram-negative bacteria. In some embodiments, the inactivated bacteria comprising an immunostimulant, the live bacteria comprising an immunostimulant configured to be transiently present in the blood stream of a host, and/or the immunoactive-transient-bacteria is a Gram-negative bacterium; or the bacteria-derived particle comprising an immunostimulant is derived from a Gram-negative bacterium.

In some embodiments, the Gram-negative bacteria is a member of a genus selected from: Salmonella, Escherichia, Helicobacter, Neisseria, Yersinia, and Pseudomonas. In some embodiments, the Gram-negative bacteria is selected from: Salmonella enterica subsp. typhimurium (“S. tymphimurium”), Escherichia coli, Helicobacter pylori, Neisseria meningitidis, Yersinia pestis, and Pseudomonas aeruginosa. In some embodiments, the Gram-negative bacterium is a member of the Salmonella genus and is a Salmonella typhimurium. In some embodiments, the S. typhimurium has an attenuated or deleted stm3120 gene.

In some embodiments, the tumor-homing is further genetically modified to express one or more tumor antigens and/or one or more immunomodulators. In some embodiments, the bacterial adjuvant is further genetically modified to express one or more tumor antigens and/or one or more immunomodulators. In some embodiments, the immunoactive-transient-bacteria is further genetically modified to express one or more tumor antigens and/or one or more immunomodulators. In some embodiments, the one or more tumor antigens comprise a neoantigen.

In some embodiments, the immunoactive-transient-bacteria is present at a higher dose than the tumor-homing bacteria. In some embodiments, the dose of the immunoactive-transient-bacteria relative to the dose of the tumor-homing bacteria is at least 8:1. In some embodiments, the dose of the immunoactive-transient-bacteria relative to the dose of the tumor-homing bacteria is at least 10:1. In some embodiments, the dose of the immunoactive-transient-bacteria relative to the dose of the tumor-homing bacteria is at least 20:1. In some embodiments, the dose of the immunoactive-transient-bacteria relative to the dose of the tumor-homing bacteria is at least 30:1. In some embodiments, the dose of the immunoactive-transient-bacteria relative to the dose of the tumor-homing bacteria is at least 50:1. In some embodiments, the dose of the immunoactive-transient-bacteria relative to the dose of the tumor-homing bacteria is at least 100:1. In some embodiments, the dose of the immunoactive-transient-bacteria relative to the dose of the tumor-homing bacteria is at least 200:1. In some embodiments, the dose of the immunoactive-transient-bacteria relative to the dose of the tumor-homing bacteria is more than 250:1. In some embodiments, the dose of the immunoactive-transient-bacteria relative to the tumor-homing bacteria is from 500:1 to 10:1. In some embodiments, the dose of the immunoactive-transient-bacteria relative to the dose of the tumor-homing bacteria is from 400:1 to 20:1. In some embodiments, the dose of the immunoactive-transient-bacteria relative to the dose of the tumor-homing bacteria is from 300:1 to 30:1.

### Methods and Uses of the Invention

In some embodiments, the tumor-homing bacteria, host-transient bacterial adjuvant, and/or immunoactive-transient-bacteria is formulated in a composition. In some embodiments, the tumor-homing bacteria and the host-transient bacterial adjuvant are in separate compositions. In some embodiments, the tumor-homing bacteria and the host-transient bacterial adjuvant are formulated in a single composition. In some embodiments, the tumor-homing bacteria and the immunoactive-transient-bacteria are in separate compositions. In some embodiments, the tumor-homing bacteria and the immunoactive-transient-bacteria are formulated in a single composition.

The present disclosure provides a method of modulating an immune system response and enhancing tumor-homing in a subject, comprising parenteral administration of any pharmaceutical combination disclosed herein.

In some embodiments, there is provided method of modulating an immune system response and enhancing tumor homing comprising a pharmaceutical combination disclosed herein. In some embodiments, the method comprises administering a dose of tumor-homing bacteria that is at least 50% less than the tumor-homing bacteria's maximum tolerated dose (MTD). In some embodiments, the dose of tumor-homing bacteria is at least 60% less than the tumor-homing bacteria's MTD. In some embodiments, the dose of tumor-homing bacteria is at least 70% less than the tumor-homing bacteria's MTD. In some embodiments, the dose of tumor-homing bacteria is at least 80% less than the tumor-homing bacteria's MTD. In some embodiments, the dose of tumor-homing bacteria is at least 90% less than the tumor-homing bacteria's MTD. In some embodiments, the dose of tumor-homing bacteria is at least 95% less than the tumor-homing bacteria's MTD.

In some embodiments, there is provided a method of modulating an immune system response and enhancing tumor-homing comprising a pharmaceutical combination disclosed herein. In some embodiments, the method comprises administering to a host circulatory system a host-transient bacterial adjuvant comprising a supplementary dose of an immunostimulant, or derivative or precursor thereof, and a tumor-homing bacteria constitutively expressing an immunostimulant, or a derivative or precursor thereof. In some embodiments, the host-transient bacterial adjuvant presents to a host the immunostimulant, derivative, or precursor thereof, for a limited period of time. In some embodiments, the derivative of the immunostimulant is a toxicity-reduced derivative of the immunostimulant.

In some embodiments, the method comprises prevention, delay of progression, or treatment of a cancer.

In some embodiments, there is provided a method of preventing, delaying the progression of, or treating a cancer comprising administering to a subject a pharmaceutical combination disclosed herein.

In some embodiments, the method comprises parenteral administration of the pharmaceutical combination In some embodiments, the method comprises parenterally administering a dose of tumor-homing bacteria and parenterally administering a host-transient bacterial adjuvant comprising a supplementary dose of an immunostimulant, or a derivative or precursor thereof; waiting a period of time for tumor-homing to occur; and administering a second anti-cancer agent or treatment.

In some embodiments, the tumor-homing bacteria and the immunoactive-transient-bacteria are simultaneously administered. In some embodiments, the tumor-homing bacteria and the immunoactive-transient-bacteria are sequentially administered. In some embodiments, the tumor-homing bacteria and the host-transient bacterial adjuvant are simultaneously administered. In some embodiments, the tumor-homing bacteria and the host-transient bacterial adjuvant are sequentially administered.

In some embodiments, the parenteral administration is by an intratumoral, intra-peritoneal, or intravenous route. In some embodiments, the pharmaceutical combination is administered by intravenous route. In some embodiments, the tumor-homing bacteria is administered by intratumoral route, and the immunoactive-transient-bacteria is administered by intravenous route.

In some embodiments, the immunoactive-transient-bacteria is an immunoactive inducible strain and the pharmaceutical combination does not comprise an inducer or comprises trace amounts of an inducer. In some embodiments, the immunoactive-transient-bacteria is in an induced state in formulation without the presence of an inducer.

In some embodiments, the immunoactive-transient-bacteria and/or the tumor-homing bacteria is genetically modified to produce a toxicity-reduced immunostimulant.

In some embodiments, the method further comprises administration of a second agent or treatment. In some embodiments, the second agent or treatment is an anti-cancer agent or treatment. In some embodiments, the anti-cancer agent or treatment comprises an immunotherapy. Various anti-cancer immunotherapies are known in the art and include, e.g., CTLA-4, PD-1, PD-L1, PD-L2, GITR, 4-1BB, CD40, and OX40. In some embodiments, the second anti-cancer agent is an immunotherapy. In some embodiments, the immunotherapy is a PD-1 inhibitor.

The second anti-cancer agent or treatment may be any appropriate treatment or agent known in the art. For example, the anti-cancer agent or treatment may comprise a cell cycle-synchronizing agent, chemotherapeutic agent, cytotoxic agent, ligand for cellular receptor(s), targeted therapy agent, immunomodulatory agent, pro-apoptotic agent, anti-angiogenic agent, cytokine, growth factor, antibody or antigen-binding fragment thereof, cell therapy, antigen, or combinations thereof.

### Tumor-Homing Bacteria: Immunoactive-Constitutive-Bacteria

As used herein, the tumor-homing bacteria is an immunoactive-constitutive-bacteria unless stated otherwise. The term immunoactive-constitutive-bacteria is meant to relate to the bacteria's ability to continuously present an immunostimulant, or a derivative or precursor thereof. In some embodiments, the tumor-homing bacteria is in its native form with respect to the immunostimulant. In some embodiments, the tumor-homing bacteria is genetically modified to produce a toxicity-reduced immunostimulant.

In some embodiments, the dose of the immunoactive-transient-bacteria is significantly greater than the dose of the tumor-homing bacteria or the immunoactive-constitutive-bacteria. A significantly great dose may refer to at least 2 times, at least 10 times, at least 100 times, or at least 1000 times the dose of the tumor-homing bacteria.

### Tumor-Homing Bacteria: Genetic Modifications for Increased Human Serum Viability

In some embodiments, the tumor-homing bacteria is genetically modified for reduced toxicity and/or increased human serum viability. Human serum viability is an indicator for increasing the potential for tumor colonization in humans. Therefore, in some embodiments, at least a portion of the pharmaceutical combination includes a tumor-homing bacteria which is an immunoactive expression-constitutive bacteria that is not genetically modified to have transient synthesis of the immunostimulant.

In some embodiments, the immunoactive expression-constitutive bacteria is in its natural state with respect to the synthesis of the immunostimulant. In some embodiments, the immunoactive expression-constitutive bacteria is genetically modified for reduced toxicity of the immunostimulant.

In some embodiments, the tumor-homing bacteria or immunoactive-constitutive-bacteria exhibits a percent viability in human serum at 3 hours of more than 3-fold relative to Salmonella typhimurium VNP20009. In some embodiments, the percent viability of the tumor-homing bacteria in human serum at 3 hours is more than 0.01%. In some embodiments, the percent viability of the tumor-homing bacteria in human serum at 3 hours is more than 0.1%. In some embodiments, the percent viability of the tumor-homing bacteria in human serum at 3 hours is more than 0.2%. In some embodiments, the percent viability of the tumor-homing bacteria in human serum at 3 hours is more than 0.5%. In some embodiments, the percent viability of the tumor-homing bacteria in human serum at 3 hours is more than 1%. In some embodiments, the percent viability of the tumor-homing bacteria in human serum at 3 hours is more than 3%. In some embodiments, the percent viability of the tumor-homing bacteria in human serum at 3 hours is more than 5%. In some embodiments, the percent viability of the tumor-homing bacteria in human serum at 3 hours is more than 7%. In some embodiments, the percent viability of the tumor-homing bacteria in human serum at 3 hours is more than 10%.

### Host-Transient Bacterial Adjuvant Comprising a Supplementary Dose of an Immunostimulant, or a Derivative or Precursor Thereof

It was expected that the presence of an immunostimulant, distinguished from the tumor-homing bacteria, which by itself produces immunostimulation, would amplify the negative side effects which typically limit parenteral dosing. It was unexpectedly found that the efficacy of a tumor-homing bacteria could be improved by the addition of a supplementary dose of an immunostimulant, while still retaining safety at an acceptable level.

Unexpectedly, the transient presence in the blood of the host-transient bacterial adjuvant or the immunoactive-transient-bacteria increases the efficacy of the tumor-homing bacteria.

The “host-transient bacterial adjuvant comprising a supplementary dose of an immunostimulant, or a derivative or precursor thereof” may be characterized by transient toxicity in a host blood stream as measured by a return of a toxicity measure to basal levels in a host body at a second time point post administration, e.g., 6 hours post administration. The host-transient bacterial adjuvant may also be characterized by transient toxicity in a host blood stream as measured by a significant reduction of the immunostimulant in the host blood stream at a second time point post administration, e.g., 6 hours post administration. Without being limited by theory, significant reduction in the blood stream and/or a return of a toxicity measure to basal levels is expected to correlate with clinical recovery associated with bacterial injection. It may be measured relative to the corresponding host-constitutive supplementary dose of immunostimulant which has not been adapted for significant reduction in the blood stream of a host at a post-administration time point, e.g., 6 hours post administration.

Calculation of a reduction of the immunostimulant in the host blood stream may be based on methods for quantifying the presence of an immunostimulant in the blood that are known in the art, such as quantifying LPS activation levels (LAL). For example, if the immunostimulant is LPS or a toxicity-reduced derivative thereof, endotoxin activity correlated with LPS activation levels in the blood of mice following injection can be quantified. The Limulus Amebocyte Lysate (LAL) assay uses blood from the horseshoe crab to detect very low levels of LPS. The presence of LPS will result in coagulation of the limulus blood lysate due to amplification via an enzymatic cascade. Gel clotting, turbidometric, and chromogenic forms of the LAL assay are commercially available. In some embodiments, LPS is significantly reduced in the blood stream of a host at a second time point post administration, e.g., 6 hours post administration.

The host-transient bacterial adjuvant comprising a supplementary dose of an immunostimulant, or a derivative or precursor thereof, comprises an immunostimulant, derivative, or precursor that is presented separately from the immunostimulant present in the tumor-homing bacteria. In some embodiments, the bacterial adjuvant or immunoactive-transient-bacteria and the tumor-homing bacteria each express one or more immunostimulants which form part of the same or distinct classes of immunostimulants. In some embodiments, the bacterial adjuvant or immunoactive-transient-bacteria expresses one or more immunostimulants which is identical or distinct from the immunostimulant provided in the tumor-homing bacteria. In some embodiments, the bacterial adjuvant or immunoactive-transient-bacteria provides for a pyrogenic activity increase of about 70%, about 75%, about 80%, about 85%, about 90%, or about 95% relative to the tumor-homing bacteria.

In some embodiments, the bacterial adjuvant comprises an outer membrane molecule (OMV), fimbriae, pili, lipopeptide, or lipoprotein. For example, the bacterial adjuvant may comprise a Lipid A, LPS, flagellin, lipoprotein, fimbriae, peptidoglycan, LTA, core oligosaccharide, or O-antigen. Additional suitable molecules may be identified by a rabbit method, well known in the art, involving assessment of rectal temperature after intravenous administration of the immunostimulant.

In some embodiments, the bacterial adjuvant or immunoactive-transient-bacteria clears a host body at a faster rate than a corresponding bacterial adjuvant or immunoactive-transient-bacteria which has not been adapted for significant reduction of its levels in the blood stream of a host at a second time point post administration, e.g., at 6 hours post administration.

The configuration of a bacterial adjuvant or immunoactive-transient-bacteria for significant reduction in the blood stream of the immunostimulant in a host at a post-administration time point, e.g., 6 hours post administration, may encompass a suicide bacteria or auxotroph bacteria which is genetically programmed for death at a delayed timepoint. An “auxotroph bacteria” refers to a recombinant bacteria which cannot grow in the absence of at least one exogenously provided essential amino acid or a precursor thereof. In one example, a recombinant bacteria can be generated by, e.g., deletion of the aspartate-beta-semialdehyde dehydrogenase gene (asd) so the bacteria cannot grow in the absence of exogenous meso-2,6-diaminopimelic acid. A suicide bacteria refers to a bacteria having an inducible genetic suicide mechanism such as a heterologous endonuclease. In some embodiments, the auxotrophy is due to a deletion or inactivating mutation in an essential metabolic gene such as the dapA gene or its functional homologs. In another example, synthesis of the immunostimulant is dependent on the presence of a riboswitch, inducer, or repressor. In some embodiments, synthesis of the immunostimulant is altered using RNA interference or other techniques. In some embodiments, protein function is altered by providing inhibitors (e.g., synthetic or natural competitive or non-competitive ligands, antibodies, etc.).

In some embodiments, the immunostimulant is an immunoactive-transient-bacteria.

### Immunoactive-Transient-Bacteria

In some embodiments, the immunoactive-transient-bacteria is a bacteria genetically modified for transient synthesis of an immunostimulant comprising PAMPs. Genetic modification may be achieved through the deletion or alteration of a gene responsible for encoding an enzyme or protein integral to the synthesis of said immunostimulatory compound or by incorporation of an additional gene for expression that facilitates transient synthesis of the immunostimulatory compound via an associated biological mechanism.

In some embodiments, the immunoactive-transient-bacteria is genetically modified for transient synthesis of an immunostimulant, said genetic modification comprising a deletion or modification in the gene encoding a protein involved in synthesis of the immunostimulant, and further insertion of a gene encoding a protein involved in synthesis of the immunostimulant and genetic association of said gene with a mechanism for transient synthesis of the immunostimulant. In some embodiments, the genetic association of said gene with a mechanism for transient synthesis of the immunostimulant is with an inducible prokaryotic promoter.

As used herein, transient synthesis of the immunostimulant refers to the enhanced synthesis of an immunostimulant which is substantially or entirely halted at a controlled timepoint. The synthesis may be induced or accelerated during a specific and defined time period, and may also allow for synthesis or presence of the immunostimulant at low levels outside that period; for example, when in an uninduced state under control of a leaky promoter. For example, synthesis may be specifically stimulated by an external factor (e.g., a specific trigger, event, or condition), resulting in a temporary peak of expression, while maintaining a baseline level of activity during other periods. In cases where the immunoactive-transient-bacteria is an immunoactive inducible strain, the immunostimulant may be produced to a minimal level even upon removal of the inducer due to leaky expression.

In some embodiments, “transient synthesis of the immunostimulant” refers to a genetic modification comprising the insertion of an immunostimulant under control of an inducible prokaryotic promoter. In some embodiments, the genetic modification comprises a deletion or attenuation in a gene encoding a protein involved in synthesis of the immunostimulant, insertion of a gene encoding a protein involved in the synthesis of the immunostimulant, and genetic association of said inserted gene with a mechanism for transient synthesis of the immunostimulant. The gene encoding a protein involved in synthesis of the immunostimulant in the context of deletion or attenuation is the same gene which is subsequently reinserted. The transient synthesis of the immunostimulant is an alternative mechanism for reducing toxicity wherein the genetic modification results in DNA level reduction in expression or RNA level reduction of translation of a functional immunostimulant after a transient period of synthesis post administration.

In some embodiments, an inducible prokaryotic promoter or alternative facilitator of transient synthesis provides for leaky expression of the immunostimulant when not exposed to the inducer.

In some embodiments, the immunoactive-transient-bacteria is genetically modified for reduced toxicity of a native immunostimulant. In some embodiments, the genetic modification for reduced toxicity comprises a genetic modification of a gene related to biosynthesis of the immunostimulant. The effect may be a post-synthesis toxicity reduction by structural modification of the immunostimulant compared with a bacteria without the genetic modification.

In some embodiments, the immunoactive-transient-bacteria comprises a live bacteria transiently present in the blood stream of a host. For example, the live bacteria may be a viable bacteria genetically modified to downregulate biosynthesis of an immunostimulant sufficient to substantially reduce toxicity. In another example, the live bacteria may include a disruption or deletion of the aspartate-semialdehyde dehydrogenase (asd gene), which permits growth in DAP supplemented medium but limits replication in vivo when administered to subjects for treatment. Such bacteria will be transiently present in the blood stream upon administration.

In some embodiments, the immunoactive-transient-bacteria is a live bacteria genetically modified for transient biosynthesis or down-regulation of the immunostimulant. This live bacteria may synthesize an immunostimulant which retains a core oligosaccharide domain and/or an O-antigen domain predicted to react with TLR4 in humans, and takes advantage of the natural heterogeneity and diversity of lipid A in the bacterial world.

In some embodiments, the immunoactive-transient-bacteria belongs to a species known for tumor colonization when administered by injection to a host with a tumor.

In some embodiments, the immunoactive-transient-bacteria is genetically modified for reduced toxicity. This may refer to a genetic modification which provides attenuated activity in a host of TLR4, TLR3, TLR1, TLR7, TLR5, or TLR2, reduced TNF-alpha and/or IFN-gamma secretion, and/or reduced complement activation, compared to activity of bacteria without the genetic modification in the host.

In some embodiments, the immunoactive-transient-bacteria, as disclosed herein, does not contain DNA that encodes or expresses non-bacterial proteins (e.g., tumor antigens or immunomodulators). In some embodiments, the immunoactive-transient-bacteria, bacterial adjuvant, or tumor-homing bacteria, as disclosed herein, contains DNA that encodes or expresses non-bacterial proteins (e.g., tumor antigens or immunomodulators).

### Genetic Modifications for Reducing Toxicity of an Immunostimulant for Either Tumor-Homing Bacteria or Immunoactive-Transient-Bacteria

In some embodiments, the tumor-homing bacteria is genetically modified for reduced toxicity in a host. In some embodiments, the reduced toxicity comprises the reduction of a pro-inflammatory immune response in a host compared to an inflammatory response induced by a corresponding wild-type bacteria, i.e., without the genetic modification. In some embodiments, despite the reduced toxicity, there is leaky expression of the immunostimulant. In some embodiments, the bacterial adjuvant or immunoactive-transient-bacteria, and/or the immunoactive expression-constitutive bacteria are genetically modified to reduce toxicity of the immunostimulant. Suitable genetic modifications are known in the art in the context of providing a degree of attenuation/reducing toxicity that is safe for intravenous injections. As used herein, genetic modification for reducing toxicity refers to any genetic modification known in the art which may provide for attenuated activity in a host of TLR4, TLR3, TLR1, TLR7, TLR5, or TLR2, reduced TNF-alpha induction or secretion, reduced IFN-gamma induction or secretion, and/or reduced complement activation, compared to activity of a bacteria without the genetic modification in the host. In some embodiments, the genetic modification for reduced toxicity refers to any genetic modification known in the art which provides for attenuated activity in a host of TLR4 or TLR5.

In some embodiments, the genetic modification for reducing toxicity of the tumor-homing bacteria, bacterial adjuvant, and/or immunoactive-transient-bacteria comprises attenuated or absent activity in a host of TLR5 compared to a bacteria without the attenuation. Examples include bacteria with attenuated or absent flagella such as fliC⁻ and fljB⁻ mutants, which are non-flagellated and induce less TLR5-mediated inflammation.

In some embodiments, the genetic modification for reducing toxicity of the tumor-homing bacteria, bacterial adjuvant, and/or immunoactive-transient-bacteria comprises attenuated or absent activity of the lpxM gene which consequently diminishes TLR4 activation. The lpxM gene encodes a late acyltransferase in the Lipid A biosynthetic pathway. Deleting lpxM results in tetra-acylated Lipid A, which decreases TLR4 activation.

In some embodiments, the genetic modification for reduced toxicity comprises a genetic modification of one or more genes involved in biosynthesis or modification of the immunostimulant, e.g., a cell surface molecule, e.g., LPS. For example, the genetic modification may comprise a process of down-regulating a gene involved in rendering the lipid A portion of LPS toxic or a process of up-regulating a gene involved in rendering the lipid A portion of LPS less toxic.

Another example relates to insertion of LpxE, which encodes for an inner membrane Lipid A 1-phosphatase from Francisella tularensis which can selectively remove the 1-phosphate group from lipid A in Salmonella, producing 1-dephosphorylated lipid A which is a close analog of the clinically approved adjuvant monophosphoryl lipid A (MPL) that remains covalently linked to LPS. Wild-type Lipid A activates both the TLR4-TRIF and TLR4-MyD88 pathways, whereas MPL selectively activates the TLR4-TRIF signaling pathway, leading to significantly lower secretion of proinflammatory cytokines such as IL-6, IL-1β, and IFN-γ, and robust induction of G-CSF, MCP-1, and IP-10, compared to bacteria without the insertion.

Examples of genes involved in the biosynthesis of an immunostimulant such as lipopolysaccharide are known in the art and may be selected from msbB, htrB, pagL, pagP, lpxR, arnJ, ep/A, and lpxT. In some embodiments, the gene involved in the biosynthesis of an immunostimulant is not involved in the biosynthesis of flagellin (i.e., flaA, flaB, fliC, fljB, fljD, fljS, rfaL, rfaG, rfaHI, rfaD, rfaP).

Biosynthesis may refer to a post-synthesis toxicity reduction by structural modification of the immunostimulant. The structural modification may relate to a structural modification in Lipid A, LPS, flagellin, lipoprotein, fimbriae, peptidoglycans, LTA, core oligosaccharide, or O-antigen. For example, the structural modification may relate to a Lipid A structural modification.

The structural modification may be provided by upregulation (i.e., genetic insertion) or downregulation (i.e., genetic attenuation [e.g., replacement]) of one or more genes involved in lipid A structural modifications. Examples include replacement, insertion, attenuation, or deletion of one or more endogenous or exogenous genes involved in LPS structural modifications. Further examples include insertion, attenuation, or deletion of a gene selected from: pagE, pagP, pagL, lpxM, lpxL, lpxE, lpxR, lpxF, and lpxT. In some embodiments, the structural modification is provided by insertion and expression of lpxXL, lpxF, or lpxE. In some embodiments, the gene involved in lipid A modification is Lipid A 1-phosphatase (lpxE), which generates monophosphorylated lipid A. lpxM provides for synthesis of tetra-acylated lipid A; insertion of lpxL provides for a reduced deacylation tetraacylated form of Lipid A; lpxE provides for a monophosphorylated lipid A; and lpxR provides for a deacylated lipid A. Insertion, attenuation, or deletion of any of these genes provides for a toxicity-reduced form of the Lipid A portion of the lipopolysaccharide molecule compared to lipid A molecules in a corresponding “wild-type” bacteria (with respect to the modified gene).

Combinations of Tumor-Homing Bacteria and/or Immunoactive-Transient-Bacteria

A tumor-homing bacteria is any bacteria parenterally administered to a host which exhibits a high level of homing to a tumor site. Homing to a tumor site may include a tumor and/or tumor microenvironment and/or tumor-resident immune cell colonization. Typically, the tumor-homing bacteria continuously produces or presents an immunostimulant. This constitutive expression can limit parenteral dosing. Unless otherwise indicated, the tumor-homing bacteria disclosed herein constitutively produces or presents an immunostimulant, or a derivative or precursor thereof.

As used herein, “engineered for tumor homing” or “configured for tumor homing” is meant to include genetically engineered bacteria which result in at least one of: increased serum half-life compared to its parental strain; increased numbers of colony forming units within the solid tumor compared to its parental strain; and reduced immune elimination following repeated dosing compared to its parental strain. In some embodiments, “engineered for tumor-homing” comprises a genetically engineered bacteria which results in an increased number of colony forming units within a solid tumor of a host compared to its parental strain. In cases where the parental strain causes host mortality, any increase in number of colony forming units, increased serum half-life, or a reduced immune elimination may represent effective tumor-homing.

Typically, the tumor-homing bacteria has been virulence attenuated for safe injection to a host with a tumor. This may involve deletion of bacterial effector proteins having virulence activity which are transported by one or more bacterial proteins.

One of the reasons for the failure in the clinical trial for S. typhimurium VNP20009 was due to lack of sufficient tumor colonization. In some embodiments, the dose of the tumor-homing bacteria is substantially less than a maximum tolerated dose for a given tumor-homing bacteria, e.g., 50% less than the MTD. “Substantially less than a maximum tolerated dose” may refer to less than 50%, less than 40%, less than 30%, less than 20%, less than 10%, less than 5%, less than 3%, less than 1%, less than 0.5%, or less than 0.05% of a maximum tolerated dose for a given tumor-homing bacteria. In some embodiments, the dose is less than 50%, less than 40%, less than 30%, less than 10%, less than 5%, less than 3%, less than 1%, less than 0.5%, or less than 0.05% of a maximum tolerated dose for a given tumor-homing bacteria.

In some embodiments, the dose of the tumor-homing bacteria is substantially less than a maximum tolerated dose of S. typhimurium VNP20009. “Substantially less than a maximum tolerated dose of S. typhimurium VNP20009” refers to less than 90%, less than 80%, less than 70%, less than 60%, less than 50%, less than 40%, less than 30%, less than 20%, less than 10%, less than 5%, or less than 3% of the maximum tolerated dose of S. typhimurium VNP20009 (e.g., 10⁸ CFU/m²) or the tumor-homing bacteria. In some embodiments, the dose of the tumor-homing bacteria is substantially less than a maximum tolerated dose of S. typhimurium VNP20009. In some embodiments, the dose of the tumor-homing bacteria is less than 10⁸ CFU/m². In some embodiments, the dose of the tumor-homing bacteria is less than 10⁷ CFU/m². In some embodiments, the dose of the tumor-homing bacteria is less than 10⁶ CFU/m².

In some embodiments, the tumor-homing bacteria is oncolytic.

In some embodiments, the tumor-homing bacteria comprises intact flagella. In some embodiments, the tumor-homing bacteria does not comprise a mutation in flagellin such as fliC or fljB.

The tumor-homing bacteria or immunoactive-transient-bacteria may be a Gram-negative bacterium or a Gram-positive bacterium. In some embodiments, the tumor-homing bacteria or immunoactive-transient-bacteria is a Gram-negative bacteria. Gram-negative bacteria are typically characterized by their cell wall structure. The Gram-negative bacteria may be a genus selected from: Salmonella, Escherichia, Helicobacter, Neisseria, Yersinia, or Pseudomonas. The Gram-negative bacteria may be a species selected from: Salmonella typhimurium, Escherichia coli, Helicobacter pylori, Neisseria meningitidis, Yersinia pestis, or Pseudomonas aeruginosa. The tumor-homing bacteria or immunoactive-transient-bacteria may be the same or different in terms of the genus and/or species. In some embodiments, the Gram-negative bacteria is S. typhimurium.

The tumor-homing bacteria may be selected due to its tumor-homing capabilities or engineered for tumor homing and colonization and/or improved tumor-homing. For example, the tumor-homing bacteria may be a S. typhimurium with an attenuated or deleted stm3120 gene.

As used herein, S. typhimurium VNP20009 is a modified attenuated strain of S. typhimurium, which contains deletions in msbB and purl, and was generated from wild-type strain ATCC #14028. The modified attenuated strain was deposited with the American Type Culture Collection (ATCC) and assigned Accession No. 202165.

Any of the bacteria disclosed herein may further include a nucleotide sequence encoding a heterologous protein or a fragment thereof fused in frame to the 3′ end of a nucleotide sequence encoding a delivery signal from a bacterial effector protein, wherein the nucleotide sequence encoding the delivery signal from a bacterial effector protein is operably linked to a promoter. Heterologous proteins or a fragment thereof may refer to an antigen such as a tumor-associated antigen or tumor-specific antigen (e.g., neoantigen). Heterologous proteins or a fragment thereof may refer to an immunomodulator which is transported by one or more bacterial proteins which are part of a secretion system machinery. In some embodiments, the tumor-homing bacteria is genetically modified to express one or more tumor antigens and/or one or more immunomodulators. In some embodiments, the tumor-homing bacteria, the bacterial adjuvant, and/or the immunoactive-transient-bacteria is genetically modified to express one or more tumor antigens and/or one or more immunomodulators. In some embodiments, the one or more tumor antigens comprise a neoantigen.

In some embodiments, the dose of the immunoactive-transient-bacteria relative to the dose of the immunoactive-constitutive-bacteria is less than 40:1. In some embodiments, the dose of the immunoactive-transient-bacteria relative to the dose of the immunoactive-constitutive-bacteria is less than 50:1. In some embodiments, the dose of the immunoactive-transient-bacteria relative to the dose of the immunoactive-constitutive-bacteria is less than 100:1. In some embodiments, the dose of the immunoactive-transient-bacteria relative to the dose of the immunoactive-constitutive-bacteria is less than 200:1. In some embodiments, the dose of the immunoactive-transient-bacteria relative to the dose of the immunoactive-constitutive-bacteria is less than 300:1. In some embodiments, the dose of the immunoactive-transient-bacteria relative to the dose of the immunoactive-constitutive-bacteria is between 500:1 to 10:1. In some embodiments, the dose of immunoactive-transient-bacteria relative to the dose of the immunoactive-constitutive-bacteria is between, 400:1 to 20:1. In some embodiments, the dose of immunoactive-transient-bacteria relative to the dose of the immunoactive-constitutive-bacteria is between 300:1 to 30:1.

In some embodiments, the dose of the immunoactive-transient-bacteria relative to the dose of the immunoactive-constitutive-bacteria is more than 5:1. In some embodiments, the dose of the immunoactive-transient-bacteria relative to the dose of the immunoactive-constitutive-bacteria is more than 10:1. In some embodiments, the dose of the immunoactive-transient-bacteria relative to the dose of the immunoactive-constitutive-bacteria is more than 20:1. In some embodiments, the dose of the immunoactive-transient-bacteria relative to the dose of the immunoactive-constitutive-bacteria is more than 30:1. In some embodiments, the dose of the immunoactive-transient-bacteria relative to the dose of the immunoactive-constitutive-bacteria is more than 50:1. In some embodiments, the dose of the immunoactive-transient-bacteria relative to the dose of the immunoactive-constitutive-bacteria is more than 100:1. In some embodiments, the dose of the immunoactive-transient-bacteria relative to the dose of the immunoactive-constitutive-bacteria is more than 200:1. In some embodiments, the dose of the immunoactive-transient-bacteria relative to the dose of the immunoactive expression-constitutive bacteria is more than 250:1.

Pharmaceutical Combinations with Two Bacterial Strains

Provided herein is a pharmaceutical combination comprising an immunoactive-transient-bacteria genetically modified for transient synthesis of an immunostimulant, or a derivative or precursor thereof, transient survival in human serum, or inducible synthesis of an immunostimulant; and a tumor-homing bacteria which is an immunoactive-constitutive bacteria. In some embodiments, the tumor-homing bacteria constitutively presents an immunostimulant, or a derivative or precursor thereof. In some embodiments, the derivative of the immunostimulant is a toxicity-reduced derivative of the immunostimulant.

Further provided herein is a pharmaceutical combination comprising two bacterial strains, an immunoactive inducible strain and a toxicity-reduced immunoactive-constitutive bacteria, and one or more physiologically acceptable carriers. Both bacteria may be genetically modified and both bacteria may belong to a species wherein, in their wild-type form, they synthesize an immunostimulant. In some embodiments, one or both bacteria are genetically modified for reduced toxicity of the immunostimulant. In some embodiments, the immunoactive inducible strain is further genetically modified for transient synthesis of an immunostimulant, e.g., by genetically associating synthesis of the immunostimulant with an inducible prokaryotic promoter. In some embodiments, the pharmaceutical combination does not contain an inducer.

### Dosing

In some embodiments, the invention provides a pharmaceutical combination comprising a tumor-homing bacteria and a dose of an immunoactive-transient-bacteria, wherein said tumor-homing bacteria is present in a low dose while the immunoactive-transient-bacteria is present in a higher, host-transient supplementary dose.

The expression “supplementary dose” as used herein refers to an amount which increases the total amount of immunostimulant in the pharmaceutical combination to a level capable of invoking one or more of the following effects in a subject receiving the pharmaceutical combination of the present invention: (1) a modulated cytokine production (e.g., TNF alpha and IL6) within the host organism, (2) a decrease in bacteremia at an extended post-administration time point, (3) negligible mortality rates observed in murine hosts, (4) tolerable impact on body weight, including both loss and subsequent recovery, (5) sufficient survival in human serum environment relative to wild-type, which increases potential for tumor colonization in human, (6) relief of one or more symptoms associated with cancer, including inhibition or arrest of tumor growth, tumor regression, reduction in tumor size or number, or a reduction, slow down, or complete inhibition of metastasis, (7) enhancement of an antitumor immune response which does not relate directly to a tumor, (8) increase in progression-free survival (PFS) and/or overall survival (OS) of the subject receiving the pharmaceutical combination.

In some embodiments, the invention provides a pharmaceutical combination comprising a dose of a tumor-homing bacteria and a dose of an immunoactive-transient-bacteria, wherein the dose of the tumor-homing bacteria is lower than the dose of the immunoactive-transient-bacteria as described herein. In some embodiments, the dose of each is sufficient to produce an additive therapeutic effect.

### Methods for Treating Cancer

The pharmaceutical combinations provided herein are particularly useful in a method for the prevention, delay of progression, or treatment of cancer in a subject. More specifically, provided herein are methods of modulating an immune system response and enhancing tumor colonization in a host with comprising: parenterally administering a tumor-homing bacteria and a host-transient bacterial adjuvant comprising a supplementary dose of an immunostimulant. In some embodiments, the tumor-homing bacteria is administered in a low dose which is substantially less than a maximum tolerated dose for the tumor-homing bacteria, e.g., at least 50% less than the MTD.

In another aspect, there is provided a method of modulating an immune system response and enhancing tumor colonization in a host with cancer comprising: administering an immunoactive-transient-bacteria that is present in the pharmaceutical combination in an induced state without an inducer; and a tumor-homing bacteria.

In some embodiments, the host-transient bacterial adjuvant comprising a supplementary dose of immunostimulant, or a derivative or precursor thereof, the immunoactive-transient-bacteria, and/or the tumor-homing bacteria is injected intratumorally, intra-peritoneally, or intravenously. In some embodiments, the pharmaceutical combination is injected in or near the tumor site. In some embodiments, the pharmaceutical combination is administered systemically. In some embodiments, the bacterial adjuvant or immunoactive-transient-bacteria, and the tumor-homing bacteria are injected at distinct locations. For example, the bacterial adjuvant or immunoactive-transient-bacteria may be administered by intravenous route while the tumor-homing bacteria may be administered intratumorally. In some embodiments, the host-transient supplementary dose of immunostimulant, derivative, or precursor, and the tumor-homing bacteria are injected by intravenous route.

In some embodiments, both components of the pharmaceutical combination are administered at different time points. In some embodiments, both components of the pharmaceutical combination are administered simultaneously.

In some embodiments, the immunoactive-transient-bacteria is an immunoactive inducible strain which is administered without the presence of an inducer, meaning that it is not detectable or present in trace amounts. In some embodiments, the immunoactive-transient-bacteria is an immunoactive inducible strain which is administered in a formulation which includes an inducer.

The methods described herein may further include administering a therapy in which a second anti-cancer agent or treatment is administered, distinct from the pharmaceutical combination. Administering a second anti-cancer agent or treatment may be by simultaneous or sequential administration. In some embodiments, the second anti-cancer agent is an immunotherapy affecting an immune checkpoint such as an antagonist directed to PD-1, PD-L1, or CTLA-4; or an agonist of receptors or ligands that positively modulate the host immune response such as agonists targeting GITR, CD40, or OX40 (CD134). These are typically administered intravenously, for example at a dose range of about 0.03 milligram per kilogram to about 30 milligram per kilogram every 1 to 4 weeks, depending on the specific treatment plan. The pharmaceutical combinations described herein may be used as priming therapy to the second anti-cancer agent or treatment such that the subject receives the pharmaceutical combination prior to beginning the second anti-cancer agent or treatment.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

The following examples are intended to illustrate how to make and use the compounds and methods of this invention and are in no way to be construed as limiting. Although the invention will now be described in conjunction with specific embodiments thereof, it is evident that many modifications and variations will be apparent to those skilled in the art. Accordingly, all such modifications and variations that fall within the spirit and broad scope of the appended claims are embraced herein. The examples are in no way intended to limit the scope of the disclosure.

### Examples

### Effects of LPS-Mediated Toxicity and Immune Activation

S. typhimurium and multiple LPS-modified variants of S. typhimurium were engineered for neoantigen and multiple payload expression. The bacteria strains used throughout the examples are described below and summarized in Table 1.
1. VNP20009: msbB KO, ATCC #202165, YS1646-attenuated for safety by deletion of both the msbB and purl genes. The msbB gene encodes the enzyme Lipid A biosynthesis acyltransferase, which is involved in the addition of a secondary myristoyl chain to Lipid A. Deletion of msbB results in a less toxic form of Lipid A. Deletion of the purl gene renders the bacteria auxotrophic for purines, which are present in the interstitial fluid of tumors. This is presumed to provide enrichment in the tumor microenvironment.
2. Pac-1-LPS-wt: Salmonella typhimurium genetically modified for expression of MC38 cancer neoantigen and multiple payload expression.
3. Pac-1-ΔrfaD: Attenuated LPS-rfaD KO. The rfaD gene encodes the enzyme ADP-L-glycero-D-manno-heptose-6-epimerase, which is involved in the biosynthesis of LPS, and its deletion generates LPS-deficient bacteria.
4. Pac-1-IrfaD (uninduced or induced): Inducible LPS-rfaD KO with an inducible reinserted rfaD gene. Enables controlled induction such that the rfaD gene is present during manufacturing and up until shortly after administration. This approach allows for controlled dosing of LPS-competent bacteria while providing LPS-deficient bacteria after administration.
5. Pac-1-lpxE: Attenuated LPS-Inserted LpxE gene. Enables synthesis of monophosphorylated LPS that was shown to reduce LPS endotoxic activity while retaining its immunogenicity (See, e.g., Kong Q, Six DA, Roland KL, Liu Q, Gu L, Reynolds CM, Wang X, Raetz CR, Curtiss R 3rd. Salmonella synthesizing 1-dephosphorylated lipopolysaccharide exhibits low endotoxic activity while retaining its immunogenicity. J Immunol. 2011 Jul. 1; 187 (1): 412-23. doi: 10.4049/jimmunol.1100339.). The lpxE gene encodes the enzyme lipid A 1-phosphatase, which removes the 1-phosphate group from the lipid A component of LPS.
6. Pac-1-AmsbB: Attenuated LPS-msbB KO. The msbB gene encodes the enzyme Lipid A biosynthesis acyltransferase, which is involved in the addition of a secondary myristoyl chain to Lipid A. Deletion of msbB results in a less toxic form of Lipid A.
7. Pac-1-ImsbB (induced or uninduced): Inducible LPS-msbB KO with an inducible reinserted msbB gene. Enables controlled induction such that the msbB gene is expressed up until shortly after administration. This approach allows for controlled dosing of LPS-competent bacteria while providing LPS-deficient bacteria after a short period post-administration.

TABLE 1

| | Attenuated | Neo- | LPS | |
|---|---|---|---|---|
| Strain | Salmonella | antigen | status | LPS attenuation |
| Pac-1 | yes | no | wt | — |
| backbone | | | | |
| Pac-1-LPS- | yes | yes | wt | — |
| wt | | | | |
| Pac-1-IrfaD | yes | yes | Attenuated | rfaD KO and inserted |
| | | | | inducible rfaD |
| Pac-1-msbB | yes | yes | Attenuated | msbB KO and inserted |
| | | | | inducible msbB |
| Pac-1-lpxE | yes | yes | Attenuated | Inserted lpxE gene |
| Pac-1- | yes | yes | Attenuated | rfaD KO and inserted |
| IrfaD/lpxE | | | | inducible rfaD |
| | | | | Inserted lpxE gene |

All the strains were evaluated for a variety of quantitative measures listed in Table 2 which relate to toxicity (e.g., weight loss in mice, clearance of bacteria from the blood, induction of tumor necrosis factor alpha (TNFα), and IL6 in human whole blood), efficacy in mice, and survival in human serum.

TABLE 2

| Category | Quantitative Measure |
|---|---|
| Efficacy [human] | Resilience in human serum |
| Safety | Potency |
| | Cytokine induction-human (TNF-alpha and IL-6) |
| Safety | Maximum weight loss in mice |
| Safety | Bacteremia in mice: load in blood on Day 9 |

### TNFα and IL6 Induction

Method: To measure induction of TNFα (pg/mL in whole blood), the multiple variant strains (1,000 CFU) were added to 0.2 mL of 25% whole blood in RPMI and incubated for 24 h at 37° C. PBS was added as a negative control (undetected-marked with *) and 10 μg LPS was added as a positive control. Following incubation, serum was assayed for TNFα and IL-6 with an ELISA assay.

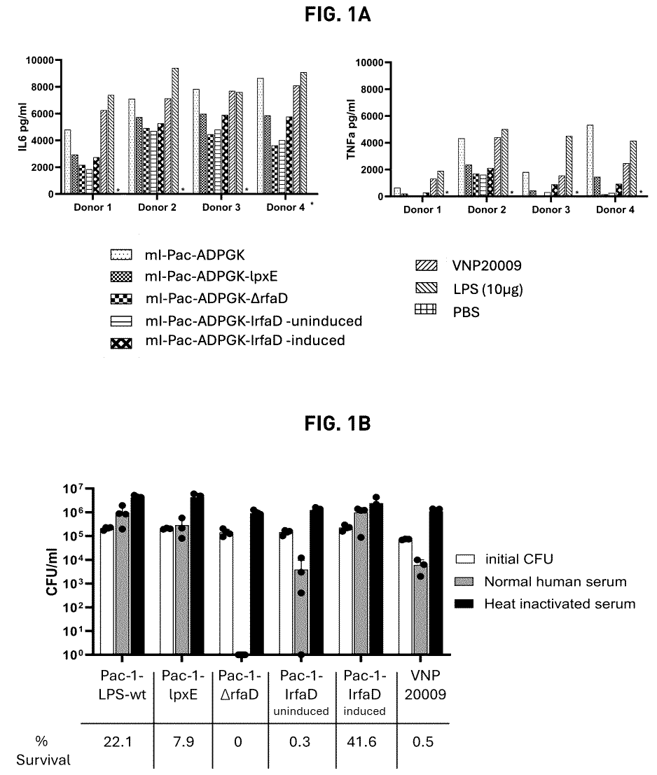

Results in FIG. 1A show similar induction levels of IL6 and TNFα for VNP20009 and mI-Pac-ADPGK, and reduced levels for LPS-attenuated strains lpxE, ΔrfaD, and inducible rfaD.

### Serum Resilience

Method: 10⁵ CFU of bacteria were incubated in 1 mL of human serum (functional complement, gray bar of FIG. 1B) or heat inactivated serum (inactivated complement, black bar of FIG. 1B) for 3 h at 37° C. Bacteria were subsequently seeded for quantification with CFU assays. Pre-incubation bacteria were also seeded for CFU to use as input controls (white bar of FIG. 1B).

Results in FIG. 1B demonstrate the ability of bacteria to survive in human serum. Percent survival, depicted below the graph, is represented as % CFU (colony forming unit) surviving compared to total CFU surviving when incubated in heat inactivated serum. Evaluation of bacteria with various LPS attenuations demonstrated full inhibition of viability for ΔrfaD strains and a significantly reduced viability for uninduced rfaD strains.

### Tumor Homing and Weight Loss

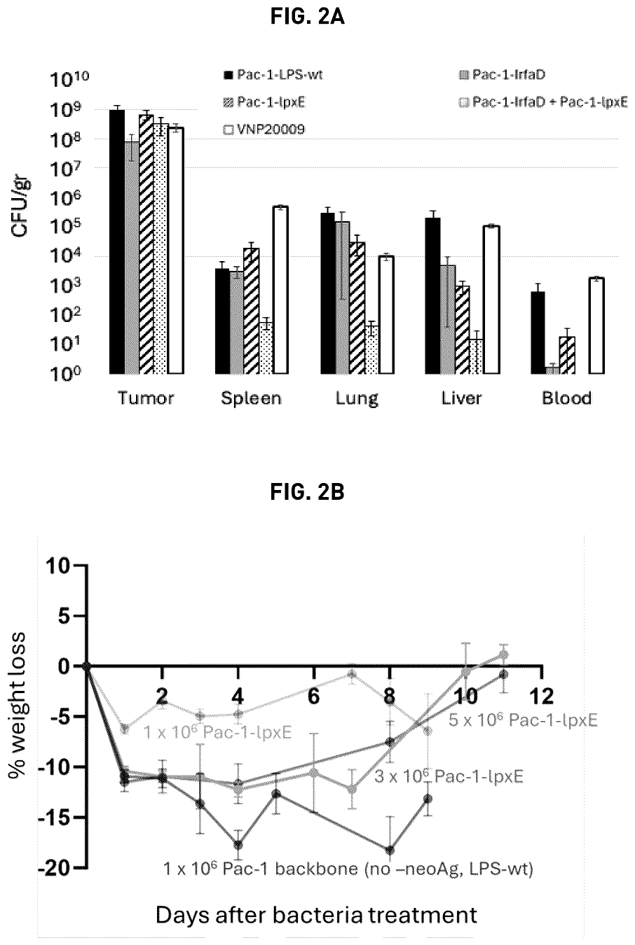

Methods: C57BL/6 mice were injected with 10⁵ MC38 cells in the right flank. When tumors reached a volume of ~100 mm³, mice received intravenous (IV) injections with 10⁶ CFU for all single injected strains and 3×10⁶ Pac-1-IrfaD and 0.1×10⁶ Pac-1-lpxE for the mixture (FIG. 2A) or at the indicated doses (FIGS. 2B-2E). Mice received 150 μg anti-PD1 by intraperitoneal (IP) injection on day 3/4 and were sacrificed on day 9 (FIG. 2A) or days 9-11 (FIG. 2B). Tumors, organs, and blood were harvested and CFU was determined. Harvested organs were vigorously shaken in 1 mL LB with a metal ball. Supernatant was serially diluted and seeded on LB plates, and then colonies were counted following 24-hour incubation at 37° C. CFU was normalized to the dilution factor and tissue mass. For blood biodistribution, 100 μL of blood was seeded on LB plates and CFU was normalized to tissue mass.

FIG. 2 demonstrates improved clearance from organs and blood, efficient tumor-homing, reduced weight loss, and increased MTD (maximum tolerated dose) of the LPS-attenuated strains.

FIG. 2A demonstrates the biodistribution of Pac-1-LPS-wt (no LPS attenuation), Pac-1-IrfaD, Pac-1-lpxE, a mixture of 3×10⁶ Pac-1-IrfaD and 0.1×10⁶ Pac-1-lpxE, or VNP20009 as evaluated in mice with MC38 tumors.

FIG. 2B shows the weight loss relative to day 0 (injection day) in mice following bacterial injection with the indicated dose of each strain. Reduced weight loss and increased recovery time is observed in mice receiving 5 million CFU of Pac-1-lpxE (lpxE attenuation), compared to 1 million of Pac-1 backbone (no neoantigen, no LPS attenuation).

FIGS. 2C-2E show reduced weight loss and/or enhanced recovery of mice following injection with various LPS-attenuated strains. The graphs show the weight loss in mice relative to day 0 (injection day) following bacterial injection of 10⁶ CFU (1M dose), 3×10⁶ CFU (3M dose), or a mixture at dose of 3×10⁶ CFU of Pac-1-IrfaD/lpxE and 10⁵ CFU of Pac-1-lpxE. The strains with different LPS attenuations (Pac-1-IrfaD, Pac-1-ImsbB, Pac-1-lpxE, or a mixture of Pac-1-IrfaD/lpxE and Pac-1-lpxE) reduce the weight loss and/or enhance the recovery of weight compared to a non LPS-attenuated strain (Pac-1-LPS-wt).

### Safety and Efficacy

Methods: For estimation of maximum tolerated dose (MTD) in human clinical trials, toxicology literature defines the upper limit for acute weight loss in short term (7-9 day) dosing studies in rodents to be 20%. Maximal weight loss is presented as % reduction in weight in mice injected with bacteria compared to day 0 (injection day). Bacterial blood levels at day 9 post injection reflect the host's clearance potential of the bacteria from the blood (measured with CFU assay, see FIG. 2A).

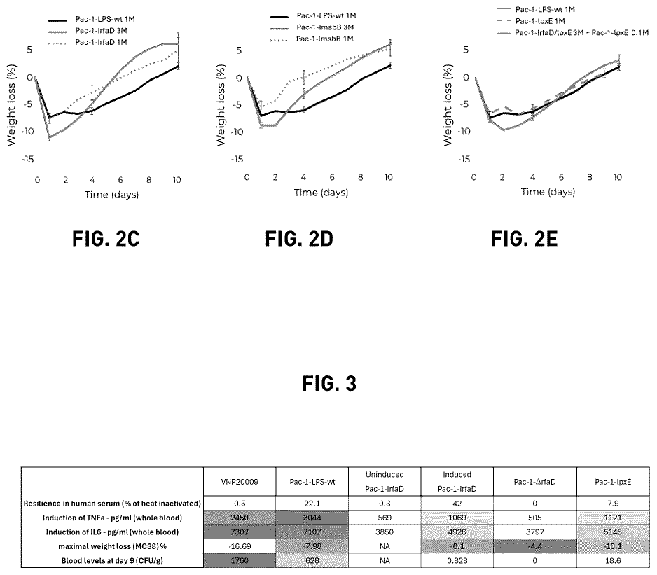

A summary of the results of various tests conducted to evaluate safety and efficacy is presented in FIG. 3, including resilience in human serum, induction of TNFα and IL6, percent maximal weight loss, and blood levels at day 9. Cells were colored using conditional formatting in Excel, which applies a grayscale gradient to the range of cells according to their values. Each row was shaded separately, and the darker shading reflects high values while the lighter shading reflects low values.

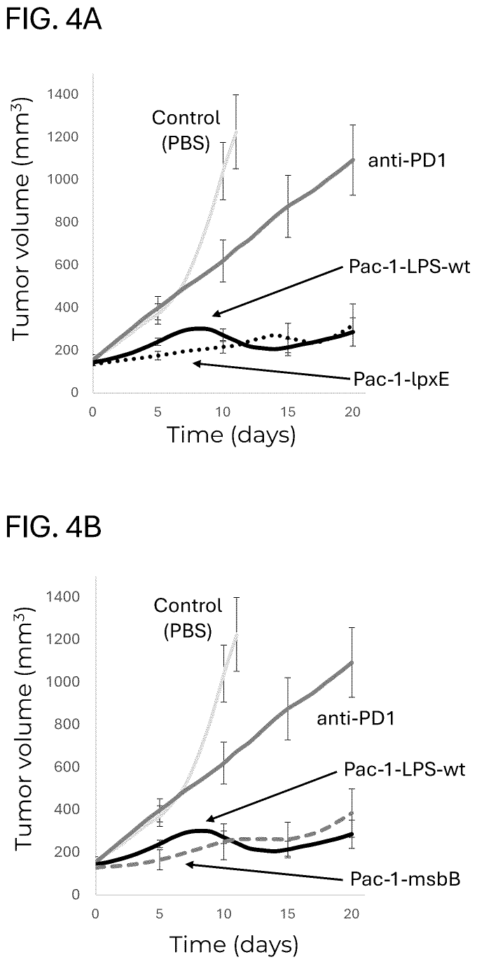

FIG. 4 demonstrates the efficacy of non-LPS-attenuated and various LPS-attenuated strains in mice with tumors. C57BL/6 mice were injected with 10⁵ MC38 cells in the right flank. When tumors reached a volume of ~100 mm³, mice received intravenous (IV) injections with the treatments described below. Mice received weekly administration of 150 μg anti-PD1 IP (PBS group was not treated), and acetylsalicylic acid (ASA) on day 3 post-bacterial injection per os (P.O., gavage, 25 mg/kg), given twice a week. The graphs in FIG. 4 show the growth of the tumors over time.
i. Light grey line: PBS only as control
ii. Dark gray line: anti-PD1
iii. Black line: 10⁶ CFU of non-LPS attenuated bacterial strain (Pac-1-LPS-wt), or
iv. Dashed Line:
   a. 3×10⁶ CFU of LPS-attenuated bacterial strain with toxicity reduction provided by lpxE (Pac-1-lpxE) (FIG. 4A),
   b. 3×10⁶ CFU of LPS-attenuated bacterial strain in induced state msbB (I-msbB; Pac-1-msbB) (FIG. 4B),
   c. 3×10⁶ CFU of LPS-attenuated bacterial strain in induced state rfaD (I-rfaD; Pac-1-IrfaD) (FIG. 4C),
   d. A mixture of 3×10⁶ LPS-attenuated bacteria in induced state rfaD with toxicity reduction provided by lpxE (I-rfaD-lpxE; Pac-1-IrfaD/lpxE) and 10⁵ tumor-homing bacteria with toxicity reduction provided by lpxE (Pac-1-lpxE) (FIG. 4D).

A significant increase in efficacy (tumor growth inhibition) is observed in MC38 mice treated with bacterial strains, which is maintained for the LPS-attenuated strains, with an improved efficacy for all strains when compared to anti-PD1 treatment only.

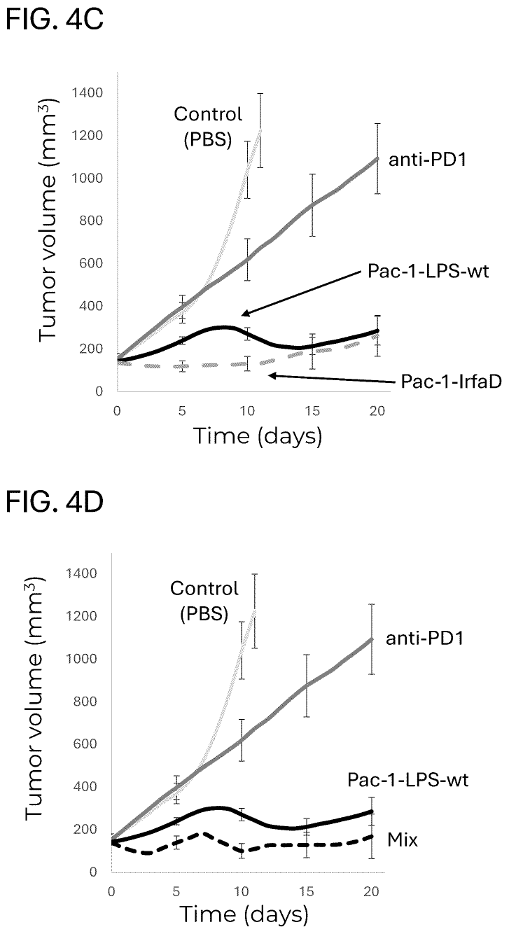

FIG. 5A shows weight loss relative to day 0 (injection day) in mice following bacterial injection. Reduced weight loss and increased recovery time is observed in mice with MC38 implanted tumors receiving 3 million CFU of Pac-1-IrfaD/lpxE+0.1 million CFU of Pac-1-lpxE, compared to 3 million of Pac-1-LPS-wt (no LPS attenuation). FIG. 5B shows the weight loss relative to day 0 (injection day) in mice following bacterial injection (3 million CFU of Pac-1-IrfaD/lpxE+0.1 million CFU of of Pac-1-lpxE). A similar pattern of reduced weight loss and increased recovery time is observed in mice implanted with various types of tumor cells.

### Lower Bacterial Doses of Tumor-Homing Bacteria Maintain Similar Tumor Colonization

Methods: C57BL/6 mice were injected with 10⁵ MC38 cells in the right flank. When tumors reached a volume of ~100 mm³, mice received two (day 0 and 11) intravenous (IV) injections with 10⁴, 10⁵, or 10⁶ CFU of Pac-1-backbone. Mice received 150 μg anti-PD1 (IP) on days 9 and 14 and were sacrificed on day 21. Tumors (T), livers (Li) and spleens (Sp) were harvested and CFU was determined. Harvested organs were chopped followed by vigorous shaking in 1 mL LB with a metal ball. Supernatant was serially diluted and seeded on LB plates and colonies were counted following 24 hr incubation at 37° C. CFU was normalized to the dilution factor and tissue mass.

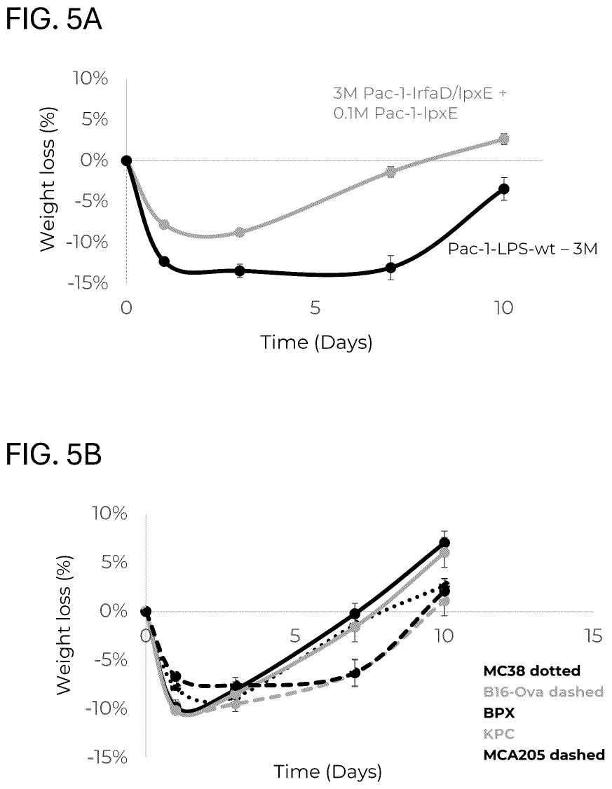

FIG. 6A shows weight loss relative to day 0 (injection day) in mice following bacterial injection at various doses. Observed weight loss in mice with MC38 implanted tumors is relative to the amount of bacteria injected to the mice, with lower weight loss observed in mice receiving 10⁴ CFU of Pac-1 backbone (no neoantigen, no LPS attenuation) compared to mice receiving higher doses of 10⁵ or 10⁶ CFU of Pac-1 backbone.

FIG. 6B shows a similar biodistribution and tumor homing capacity of Pac-1-backbone (no neoantigen, no LPS attenuation) at various dose levels when administered to MC38 tumor-bearing mice.

### Lower Bacterial Doses of Tumor-Homing Bacteria Result in Lower Anti-Tumor Efficacy

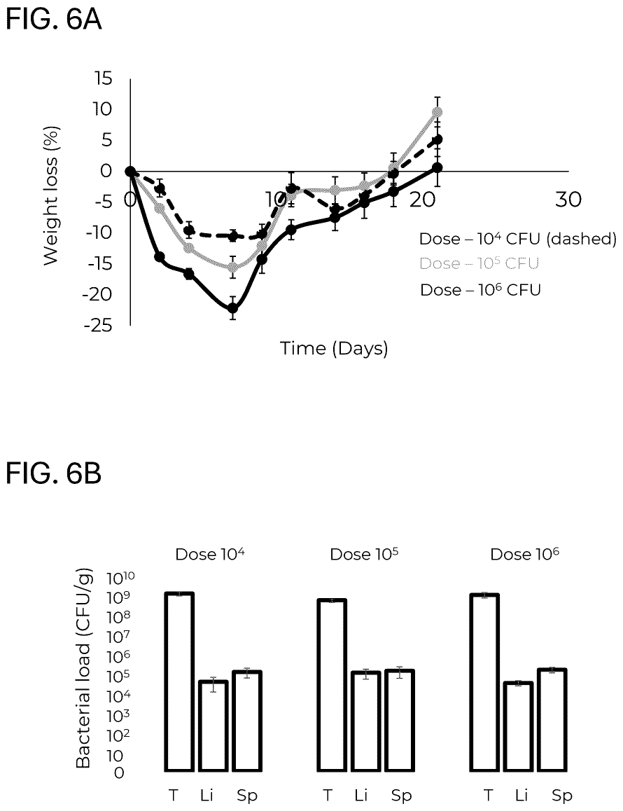

Methods: Using the MC38 tumor mouse model, the efficacy of Pac-1 variant (with neoantigen, 1 PL, LPS wt) was investigated. When tumors reached a volume of ~100 mm³, mice were injected IV (day 0) with indicated doses of Pac-1 variant. Mice received weekly administration of 150 μg anti-PD1 (IP) and ASA on day 3 post-bacterial injection per os (po, gavage, 25 mg/kg), given twice a week. Graphs show the growth of the tumors and survival of mice over time (FIGS. 7A and 7B).

A marked increase in efficacy (tumor growth inhibition and survival of mice) is observed in MC38 mice treated with the higher dose (10⁶ CFU) of Pac-1 variant bacteria.

### Combination of Low Dose of Tumor-Homing Bacteria Plus Immunostimulant Increases Efficacy

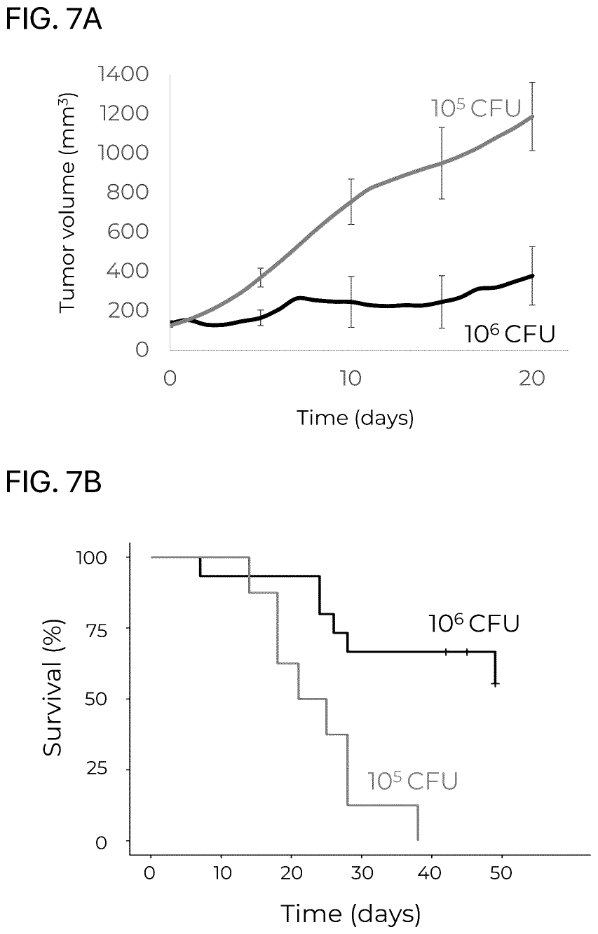

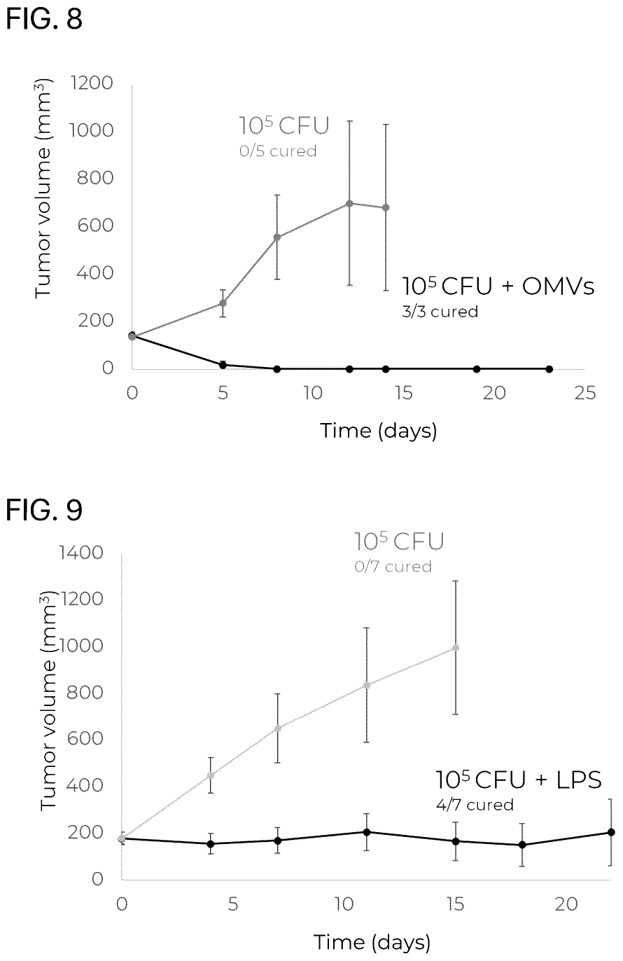

Methods: C57BL/6 mice were injected with 10⁵ MC38 cells in the right flank. When tumors reached a volume of ~100 mm³, mice received intravenous (IV) injections of 10⁵ CFU Pac-1-lpxE or Pac-1-lpxE+OMVs (10 μg by protein mass) (FIG. 8). For LPS experiments, when tumors reached a volume of ~100 mm³, mice received intravenous (IV) injections of Pac-1-lpxE or Pac-1-lpxE+LPS (50 μg/kg, from Salmonella enterica serotype typhimurium, L6511, Sigma-Aldrich) (FIG. 9). Mice received weekly administration of 150 μg anti-PD1 (IP) and ASA on day 3 post-bacterial injection per os (P.O., gavage, 25 mg/kg), given twice a week. Graphs show the growth of the tumors over time, averaged per group of mice.

To produce OMVs, fresh overnight starters were diluted and grown in LB to OD_{600 1.2}, pelleted at 5000×g for 10 min (4° C.), supernatants were filtered (0.22 micron), and OMVs were pelleted from supernatants with ultracentrifugation (150,000×g, 3 h, 4° C.), followed by a PBS wash step (150,000×g, 2 h, 4° C.). OMVs in pellets were resuspended in PBS and protein mass was quantified with Qubit.

The supplementation of a host-transient immunostimulant (OMVs) to low dose Pac-1-lpxE tumor homing bacteria demonstrates substantially improved efficacy of the treatment (FIG. 8).

The supplementation of a host-transient immunostimulant (LPS) to low dose Pac-1-lpxE tumor homing bacteria demonstrates substantially improved efficacy of the treatment (FIG. 9).

1. A pharmaceutical combination comprising:
(a) a dose of tumor-homing bacteria that is at least 50% less than the tumor-homing bacteria's maximum tolerated dose (MTD); and
(b) a host-transient bacterial adjuvant comprising a supplementary dose of an immunostimulant, or a derivative or precursor thereof.

2. The pharmaceutical combination according to claim 1, wherein the tumor-homing bacteria exhibits:
(i) constitutive toxicity in a host; and
(ii) human serum resilience at 3 hours after parenteral administration of more than 3-fold relative to Salmonella typhimurium VNP20009.

3. The pharmaceutical combination according to claim 1, wherein the host-transient bacterial adjuvant:
(i) is sufficient to provide an endotoxicity increase of at least 30% relative to the dose of the tumor-homing bacteria; and/or
(ii) exhibits a transient toxicity in a host blood stream as measured by a return of a toxicity measure to basal levels in a host at least 6 hours post administration.

4. The pharmaceutical combination according to claim 1, wherein the host-transient bacterial adjuvant is chosen from: a bacteria-derived particle comprising an immunostimulant, an inactivated bacteria comprising an immunostimulant, a live bacteria comprising an immunostimulant configured to be transiently present in the bloodstream of a host, and an immunoactive-transient-bacteria.

5. The pharmaceutical combination according to claim 1, wherein the derivative of the immunostimulant is a toxicity-reduced derivative of the immunostimulant.

6. The pharmaceutical combination according to any claim 1, wherein the pharmaceutical combination further comprises one or more physiologically acceptable carriers.

7. A pharmaceutical combination comprising one or more physiologically acceptable carriers and two bacterial strains:
(a) an immunoactive-transient-bacteria genetically modified for transient survival in human serum, inducible synthesis of an immunostimulant, or transient synthesis of an immunostimulant; and
(b) a tumor-homing bacteria which is an immunoactive-constitutive-bacteria.

8. The pharmaceutical combination according to claim 7, wherein the immunoactive-transient-bacteria is genetically modified for transient synthesis of an immunostimulant, wherein the genetic modification comprises
(i) a deletion or attenuation of a gene encoding a protein involved in synthesis of the immunostimulant; and
(ii) insertion of a gene encoding a protein involved in synthesis of the immunostimulant genetically associated with a mechanism for transient synthesis of the immunostimulant.

9. The pharmaceutical combination according to claim 8, wherein the mechanism for transient synthesis of the immunostimulant is an inducible prokaryotic promoter.

10. The pharmaceutical combination according to claim 8, wherein the mechanism for transient synthesis comprises leaky expression of the immunostimulant when not exposed to an inducer.

11. The pharmaceutical combination according to claim 1, wherein the immunostimulant is
(i) a pathogen-associated molecular pattern (PAMP) associated with bacteria; and/or
(ii) a Toll-like receptor 2 (TLR2) agonist, a Toll-like receptor 4 (TLR4) agonist, a Toll-like receptor 5 (TLR5) agonist, a Toll-like receptor 3 (TLR3) agonist, a Toll-like receptor 1 (TLR1) agonist, a Toll-like receptor 7 (TLR7) agonist, a complement activator, or a TNFα and/or IFN-γ secretion inducer.

12. The pharmaceutical combination according to claim 1, wherein the tumor-homing bacteria is oncolytic; and/or comprises intact flagella.

13. The pharmaceutical combination according to claim 1, wherein the pharmaceutical combination does not comprise an inducer.

14. The pharmaceutical combination according to claim 4, wherein:
(i) the tumor-homing bacteria is genetically modified for reduced toxicity of an immunostimulant; and/or
(ii) the immunoactive-transient-bacteria is genetically modified for reduced toxicity of an immunostimulant.

15. The pharmaceutical combination according to claim 14, wherein the genetic modification for reduced toxicity of the immunostimulant comprises a genetic modification of a gene related to biosynthesis of the immunostimulant.

16. The pharmaceutical combination according to claim 1, wherein the immunostimulant comprises Lipid A, lipopolysaccharide (LPS), flagellin, lipoprotein, fimbriae, peptidoglycan, lipoteichoic acid (LTA), core oligosaccharide, or O-antigen.

17. The pharmaceutical combination according to claim 4, wherein
(i) the tumor-homing bacteria is a Gram-negative bacterium; and/or
(ii) the inactivated bacteria comprising an immunostimulant, the live bacteria comprising an immunostimulant configured to be transiently present in the blood stream of a host, and/or the immunoactive-transient-bacteria is a Gram-negative bacterium; or the bacteria-derived particle comprising an immunostimulant is derived from a Gram-negative bacterium.

18. The pharmaceutical combination according to claim 17, wherein the Gram-negative bacterium is a member of the Salmonella genus and is a Salmonella typhimurium having an attenuated or deleted stm3120 gene.

19. The pharmaceutical combination according to claim 1, wherein the tumor-homing bacteria and/or the bacterial adjuvant is further genetically modified to express one or more tumor antigens and/or one or more immunomodulators.

20. The pharmaceutical combination according to claim 4, wherein the tumor-homing bacteria and/or the immunoactive-transient-bacteria is further genetically modified to express one or more tumor antigens and/or one or more immunomodulators.

21. The pharmaceutical combination according to claim 4, wherein the immunoactive-transient-bacteria is present at a higher dose than the tumor-homing bacteria, wherein the dose of the immunoactive-transient-bacteria relative to the dose of the tumor-homing bacteria is at least 8:1.

22. The pharmaceutical combination according to claim 1, wherein (a) and (b) are in separate compositions.

23. A method of modulating an immune system response and enhancing tumor-homing in a subject, comprising: parenteral administration of the pharmaceutical combination according to claim 1.

24. A method of modulating an immune system response and enhancing tumor-homing in a subject, comprising: parenteral administration of the pharmaceutical combination according to claim 4.

25. The method according to claim 24, wherein the tumor-homing bacteria and the immunoactive-transient-bacteria are simultaneously administered.

26. The method according to claim 24, wherein the parenteral administration is by an intratumoral, intra-peritoneal, or intravenous route.

27. The method according to claim 24, wherein the method comprises prevention, delay of progression, or treatment of a cancer.

28. The method according to claim 24, further comprising administering a second agent or treatment, wherein the second agent or treatment is an anti-cancer agent or treatment.

29. A method of modulating an immune system response and enhancing tumor-homing, comprising administering to a host circulatory system a tumor-homing bacteria constitutively expressing an immunostimulant, or a derivative or precursor thereof; and
a host-transient bacterial adjuvant comprising a supplementary dose of an immunostimulant, or a derivative or precursor thereof.

30. A method of preventing, delaying the progression of, or treating a cancer, comprising administering to a host circulatory system a tumor-homing bacteria constitutively expressing an immunostimulant, or a derivative or precursor thereof; and
a host-transient bacterial adjuvant comprising a supplementary dose of an immunostimulant, or a derivative or precursor thereof.
